(19) United States
(12) Patent Application Publication
(10) Pub. No.: US 2022/0354998 A1
(43) Pub. Date: Nov. 10, 2022

(54) HYDROGEL-FILLED MICRONEEDLE ARRAYS AND USES THEREOF

(71) Applicant: University of Connecticut, Farmington, CT (US)

(72) Inventors: Ali Tamayol, Farmington, CT (US); Lindsay Barnum, Farmington, CT (US)

(21) Appl. No.: 17/738,936

(22) Filed: May 6, 2022

Related U.S. Application Data
(60) Provisional application No. 63/185,402, filed on May 7, 2021.

(51) Int. Cl. A61L 31/14; A61M 37/00; A61L 31/16; A61L 31/04; B33Y 80/00; B33Y 40/20; B29C 64/35

(52) U.S. Cl. CPC A61L 31/145; A61M 37/0015; A61L 31/16; A61L 31/146; A61L 31/048; B33Y 80/00; B33Y 40/20; B29C 64/35; A61M 2037/0023; A61M 2037/0053; A61M 2037/0061; A61M 2037/0046; B29L 2031/7544

(57) ABSTRACT

Disclosed herein are drug delivery devices that can temporally and spatially deliver biologically active agents. An example drug delivery device includes a microneedle array comprising a plurality of microneedles on a surface of a substrate, each microneedle comprising a core comprising a hydrogel and a layer on a surface of the core. Also disclosed are methods of using the drug delivery device, and methods of making the microneedle array that includes a loading device.

### CROSS-REFERENCE TO RELATED APPLICATIONS

This application claims priority to U.S. Provisional Patent Application No. 63/185,402 filed on May 7, 2021, which is incorporated by reference herein in its entirety.

### FEDERALLY SPONSORED RESEARCH

This invention was made with government support under Grant Nos. GM126831 and AR073822 from the National Institutes of Health. The government has certain rights in the invention.

### TECHNICAL FIELD

This disclosure relates to microneedle arrays that include a hydrogel and their use in medical applications.

### BACKGROUND

Microneedle arrays are a promising technology for transdermal drug delivery and fluid sampling. They have been used in many applications ranging from insulin detection to controlled vaccine delivery. However, their use in wound healing remains largely unexplored. In clinical practice, standard of care has remained largely unchanged for decades and new approaches such as topical delivery of growth factors and autologous or artificial skin grafts produce inconsistent results. Accordingly, improved microneedle array design and fabrication strategies would be useful for broader application in the medical field.

### SUMMARY

In one aspect, disclosed are drug delivery devices that include a microneedle array comprising a plurality of microneedles on a surface of a substrate, each microneedle comprising a core comprising a hydrogel; and a layer on a surface of the core, the layer comprising a material having a compressive modulus of at least 1 MPa.

In another aspect, disclosed are methods of making a microneedle array, the method including printing a microneedle array, the microneedle array comprising a plurality of microneedles on a surface of a substrate, each microneedle comprising a first end that is coupled to the substrate, a second end opposite the first end, and a channel extending therethrough from the first end to the second end; adding a prepolymer to the channel of each microneedle; and crosslinking the prepolymer to provide a hydrogel in the channel of each microneedle.

In another aspect, disclosed are methods of treating a disorder in a subject in need thereof, the method including contacting an area of the subject's skin with a drug delivery device as disclosed herein, wherein the biologically active agent is transdermally delivered to the subject.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-E show the use of miniaturized needle arrays (MNAs—also referred to as microneedle arrays) for enhancing the bioavailability of drugs at the desired tissue depths in accordance with an embodiment of the present disclosure. FIG. 1A. Schematic representation of a MNA-based dressing for the delivery of two different therapeutics targeting different depths or regions of the tissue to target separate cell populations. FIG. 1B. A 3D-printed multi-length needle array, in which the needles of different lengths were filled with hydrogels carrying different agents (shown here with different colors). FIG. 1C and FIG. 1D. The qualitative FIG. 1C and quantitative FIG. 1D show a distribution of different dyes in an agarose skin model released from a multi-length MNAs bandage. The intensity is graphed along the lines indicated in FIG. 1C. FIG. 1E. Compliance of a semi-flexible MNA on a sample of pig skin.

FIGS. 2A-G show MNA fabrication and gel filling in accordance with an embodiment of the present disclosure. FIG. 2A. SolidWorks design of semi-flexible MNA. FIG. 2B. Schematic illustration of a fabrication process using 3D printing followed by cleaning with NaOH to remove support material. FIG. 2C. Actual demonstration of semi-flexible MNAs. FIG. 2D. Concept drawing of loading mechanism for filling MNAs. FIG. 2E. Demonstration of rhodamine dyed alginate deposition using the loading mechanism. FIG. 2F. Fluorescent imaging of filled needles. FIG. 2G. Proof-of-concept illustrating spatial control over individual needles. Two “C-like patterns in the array were filled with gels containing red and blue dyes, while the rest were filled with a transparent gel.

FIGS. 3A-H show mechanical properties and penetration testing of MNAs in accordance with an embodiment of the present disclosure. FIG. 3A. Setup employed for mechanical testing of MNAs. FIG. 3B. Force-displacement curves obtained from compression testing of 2 mm-and 3 mm-long MNAs. FIG. 3C. Needle morphology before and after compression testing. The needles bent under compression demonstrating the desired failure mechanism. FIG. 3D. Pig skin insertion testing configuration. FIG. 3E Representative force-displacement curves for pig skin insertion tests on MNAs with (i) flexible backing, (ii) non-flexible backing, and (iii) semi-flexible backing. FIG. 3F. Embedded alginate hydrogel in needles before and after insertion into pig skin. FIG. 3G. Pig skin after insertion of multi-length MNA with 3 mm needles labeled with Rh-B (red) and 2 mm needles labeled with FITC (green). FIG. 3H. Hematoxylin and eosin (H&E) staining of pig skin section that was penetrated with MNA. Black arrows indicate where needles pierced the skin.

FIGS. 4A-F show microstructure and drug delivery characterization of hydrogels embedded in MNAs in accordance with an embodiment of the present disclosure. FIG. 4A. scanning electron microscopy (SEM) micrographs of resin/alginate interface in filled needles with different magnification levels. FIG. 4B. SEM image of 1% alginate microstructure demonstrating a porous network. Release kinetics of Rh-B as a small-molecule drug model from a different concentration of alginate embedded in MNAs with 3 mm (FIG. 4C), or 2 mm needles (FIG. 4D); Release profile of bovine serum albumin (BSA) as a model protein for growth factors from alginate (FIG. 4E) and poly(ethylene glycol diacrylate) (PEGDA) (FIG. 4F), embedded in 3 mm needles. Error bars for FIG. 4F are too small to be seen after the 0 h time point.

FIGS. 5A-D show dual-drug release from multi-length MNA in accordance with an embodiment of the present disclosure. FIG. 5A Schematic representation of the experimental setup, showing the multi-length MNA inserted into the two-gel skin model. FIG. 5B MNA schematics showing the loading conditions used: both lengths blank, long needles loaded with Rh-B and short needles blank, long needles blank and short needles loaded with BSA-FITC, and long needles loaded with Rh-B and short needles loaded with BSA-FITC. Percent release of Rh-B and BSA-FITC in the top, agarose layer (FIG. 5C) and the bottom, gelatin layer (FIG. 5D).

FIGS. 6A-B show evaluation of wound healing rate in vitro using scratch assay on cultured HUVECs in accordance with an embodiment of the present disclosure. Qualitative (FIG. 6A) and quantitative (FIG. 6B) representation of gap closure in a positive control group where VEGF mixed with the culture media compared to the groups where MNAs without or with the incorporation of VEGF placed in the culture wells. Scale bars are 250 μm.

FIG. 7 shows cytocompatibility evaluation of 3D printed resin-based MNAs in accordance with an embodiment of the present disclosure. Live/Dead assay was performed after one day to indicate viable (green) and dead (red) cells, seeded on the MNA surface.

FIG. 8 shows a Solidworks drawing of a 8-needle MNA with dimensions in accordance with an embodiment of the present disclosure.

FIG. 9 shows a Solidworks drawing of a rigid 36-needle MNA with dimensions in accordance with an embodiment of the present disclosure.

FIG. 10 shows a Solidworks drawing of a semi-flexible 36-needle MNA, in accordance with an embodiment of the present disclosure, with dimensions, needle dimensions are unchanged from FIG. 9.

FIG. 11 shows a Solidworks drawing of a MNA-filling pipette attachment. in accordance with an embodiment of the present disclosure.

FIG. 12 shows a Solidworks drawing of a dual release MNA in accordance with an embodiment of the present disclosure.

FIG. 13 shows combined release data from a dual release experiment.

FIG. 14 shows SEM images of the pore structure for 1%, 2%, and 3% alginate embodiments of the present disclosure.

### DETAILED DESCRIPTION

The present disclosure provides a fabrication strategy for customizable microneedle arrays. The described microneedle arrays may utilize a 3D-printed core-and-shell approach wherein a hard resin outer shell is printed, with an end of the needle left open and a hollow core that can include a hydrogel. The disclosed microneedle arrays have robust mechanical properties that can facilitate transdermal drug delivery.

### 1. Definitions

Unless otherwise defined, all technical and scientific terms used herein have the same meaning as commonly understood by one of ordinary skill in the art. In case of conflict, the present document, including definitions, will control. Example methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “biologically active agent,” as used herein, refers to a substance that can act on a cell, virus, tissue, organ, organism, or the like, to create a change in the functioning of the cell, virus, tissue, organ, or organism. Examples of a biologically active agent include, but are not limited to, drugs, pharmaceuticals, anti-microbial agents, cells, proteins, and nucleic acids. A biologically active agent is capable of treating and/or ameliorating a condition or disease, or one or more symptoms thereof, in a subject. Biologically active agents of the present disclosure also include prodrug forms of the agent.

The term “subject” includes humans and mammals (e.g., mice, rats, pigs, cats, dogs, and horses). Typical subjects of the present disclosure may include mammals, particularly primates, and especially humans. For veterinary applications, suitable subjects may include, for example, livestock such as cattle, sheep, goats, cows, swine, and the like; poultry such as chickens, ducks, geese, turkeys, and the like, as well as domesticated animals particularly pets such as dogs and cats. For research applications, suitable subjects may include mammals, such as rodents (e.g., mice, rats, hamsters), rabbits, primates, and swine such as inbred pigs and the like.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Drug Delivery Devices

Disclosed herein are drug delivery devices that include a microneedle array. The microneedle array includes a plurality of microneedles on a surface of a substrate, where each microneedle includes a core and a layer on a surface of the core. The core includes a hydrogel. And, the layer includes a material having a compressive modulus of at least 1 MPa.

The present disclosure provides a fabrication strategy for customizable microneedle arrays. The microneedle arrays can be fabricated via 3D printing techniques, which can provide a low cost and robust strategy for device fabrication. Furthermore, the microneedle arrays and devices thereof may be used to provide the delivery of various agents under a controlled spatial and temporal distribution to a subject.

The microneedle arrays can be incorporated into a variety of transdermal drug delivery devices such as transdermal patches. For example, the substrate of the microneedle array can be attached to a backing layer to form a transdermal patch. In some embodiments, the substrate can be associated with or attached to a biologically active agent reservoir from which the biologically active agent can be delivered to the hydrogel and/or through the microneedles to a subject. The reservoir layer can be a liquid reservoir or a hydrogel reservoir or any other reservoir type known in the arts so long as the reservoir can adequately deliver the biologically active agent to the microneedles. Other material may also be incorporated into the transdermal drug delivery devices of the present disclosure such as permeation enhancers, controlled-release membranes, humectants, emollients, and the like.

A. Microneedle Arrays

As mentioned above, the microneedle array includes a plurality of microneedles. The plurality of microneedles is on a surface of the substrate. The substrate can include at least one material. The number of materials included in the substrate can be dependent on the need and the application of the microneedle array and drug delivery device thereof. For example, the substrate can be configured to conform to a biological topography (e.g., skin), such that the microneedles thereon can penetrate said biological topography and maintain their position upon penetration. In some embodiments, the substrate includes at least two different materials. In some embodiments, the substrate includes a least three different materials. In still other embodiments, the substrate includes at least four different materials.

The substrate can include materials with varying mechanical properties to allow the microneedles thereon to conform to a biological topography and to penetrate said biological topography, while also maintaining their position upon penetration. The material can include polymers (e.g., polymer resins) and/or metals. In some embodiments, the material includes a polymer. Examples of polymers include, but are not limited to, hyaluronic acid, polyethylene glycol, polypropylene glycol, polyethylene oxide, polypropylene oxide, polyglutamate, polylysine, polysialic acid, polyvinyl alcohol, polyacrylate, polymethacrylate, polyacrylamide, polymethacrylamide, polyvinyl pyrrolidone, polyoxazoline, polyiminocarbonate, polyamino acid, hydrophilic polyester, polyamide, polyurethane, poly(ethylene glycol diacrylate), poly (l-lactic acid) (PLLA), poly(d,l-lactide-co-glycolide) (PLGA), polymers of ethylene-vinyl acetates, and other acyl substituted cellulose acetates, polyurethanes, polyurea, polystyrenes, polyvinyl chloride, polyvinyl fluoride, chlorosulphonate polyolefins, poly(vinyl imidazole), poly(valeric acid), polybutyric acid, polycaprolactone (PCL), polyglycolic acid (PGA), poly(N-isopropylacrylamide), dextran, agarose, xylan, mannan, carrageenan, alginate, gelatin, collagen, cellulose, methylcellulose, ethyl cellulose, hydroxypropylmethyl cellulose, hydroxyethyl starch, chitosan, silk, derivatives thereof, co-polymers thereof, and combinations thereof. In some embodiments, the polymer is bio-absorbable or biodegradable. In some embodiments, the polymer is a printable polymer for stereolithography and/or digital light processing 3D printing. In some embodiments, the material is a metal. Examples of metals include, but are not limited to, steel, gold, titanium, and other suitable metals for drug delivery and other biomedical applications.

The substrate can include different materials in varying arrangements. For example, the substrate can include a first material and a second material, where the second material is more rigid and less flexible compared to the first material, and where the first material and the second material are in distinct regions of the substrate. In some embodiments, the substrate includes the second material as regions (e.g., islands) interconnected by the first material (e.g., ribs between the islands). In some embodiments, the microneedles are coupled to the regions of the second material. In some embodiments, an individual microneedle is coupled to an individual region of the second material. Accordingly, in some embodiments, the microneedles include the same material as the substrate. And, in some embodiments, the microneedles include at least one material of the substrate, such as the second material. In some embodiments, the substrate can include the first material having an elongation at break range of about 45% to about 55% and the second material having an elongation at break range of about 10% to about 25%. In some embodiments, the substrate can include the first material having a shore hardness of about 60 to about 62 Scale A and the second material having a shore hardness of about 83 to about 86 Scale D.

Each microneedle includes a core/shell structure. For example, the microneedle can include a first end that is coupled to the substrate, a second end opposite the first end, and a channel extending therethrough from the first end to the second end. The first end can have an opening. In addition, the second end can have an opening, which can allow substances and molecules from the core to, e.g., diffuse out from the microneedle. The channel can be the core of the microneedle. In some embodiments, the microneedle is described as being hollow. The channel can have a diameter that is sufficient in size to allow the passage of liquid and/or solid materials into and/or through the microneedle. For example, a hydrogel can be positioned within the channel. The hydrogel can be adhered to a surface of the channel. Accordingly, the core can include the hydrogel.

Each microneedle includes a layer on a surface of the core. For example, the layer can be a shell around the core extending from the first end of the microneedle to the second end of the microneedle. The layer can have a first wall and a second wall, the second wall opposite of the first wall. The first wall can be the exterior of the layer. The second wall can be the interior of the layer. The second wall can define the channel extending through the microneedle. Because the core can include a hydrogel, the layer can also be on a surface of the hydrogel.

The layer includes a material. The layer can include the same material as the substrate. Accordingly, the description of the material for the substrate can be used to describe the layer of the microneedle. For example, the material can include polymers (e.g., polymer resins) and/or metals. In some embodiments, the material includes a polymer. In addition, the material can be more rigid than the hydrogel present in the core. For example, the material can have a compressive modulus greater than that of the hydrogel, such that it is capable of penetrating tissue. The layer can provide structural support for the hydrogel. The material may have a compressive modulus of at least 1 MPa, at least 50 MPa, at least 100 MPa, at least 200 MPa, at least 300 MPa, at least 400 MPa, at least 500 MPa, at least 700 MPa, at least 1,000 MPa, or at least 2,000 MPa. In some embodiments, the material has a compressive modulus of at least 1 MPa. In some embodiments, the material has a compressive modulus of about 1 MPa to about 2,000 MPa. In some embodiments, the material of the layer is the same as the second material described above for the substrate. In some embodiments, the material of the layer has an elongation at break range of about 10% to about 25%, a shore hardness of about 83 to about 86 Scale D, or a combination thereof.

The microneedles of the disclosed microneedle array can have advantageous properties that make the arrays useful for drug delivery applications, such as transdermal drug delivery applications. For example, the microneedles can have advantageous mechanical properties that can allow them not to sustain damage upon insertion into, e.g., a wound. In some embodiments, the microneedles do not break, shatter, and/or snap off at their failure point. Rather, the microneedles can bend at their failure point. In addition, the microneedles can have a failure point at about 8% strain to about 25% strain as measured by jaw displacement, such as about 10% strain to about 22% strain, about 11% strain to about 21% strain, or about 12% strain to about 20% strain as measured by jaw displacement. Each microneedle may have a failure of at least 8% strain, at least 9% strain, at least 10% strain, or at least 11% strain as measured by jaw displacement. Each microneedle may have a failure of less than 25% strain, less than 24% strain, less than 23% strain, less than 22% strain, or less than 21% strain as measured by jaw displacement. Strain jaw displacement measurements can be made as described in the Examples.

The microneedles can have varying lengths, which can, e.g., allow the microneedles to penetrate varying depths. For example, the microneedles can have a length of about 10 μm to about 10,000 μm, such as about 50 μm to about 5,000 μm, about 100 μm to about 5,000 μm, about 10 μm to about 7,000 μm, about 150 μm to about 6,000 μm, about 500 μm to about 5,000 μm, or about 1,000 μm to about 4,000 μm. In addition, the microneedles can be oriented at an angle to the substrate or they can be configured to be perpendicular to the substrate. It is also possible to produce and provide a microneedle array which has microneedles with different angular configurations or different needle lengths.

The microneedle array can include different populations of microneedles where the different populations have different lengths, different biologically active agents, and/or different hydrogels. For example, the microneedle array can include a first plurality of microneedles, where each microneedle of the first plurality has a first length; and a second plurality of microneedles, where each microneedle of the second plurality has a second length, the second length being different from the first length. The lengths described above can be applied to the first length and the second length. The different microneedle populations can also include varying biologically active agents and/or varying hydrogel compositions. For example, the first plurality of microneedles can include a first biologically active agent, and the second plurality of microneedles can include a second biologically active agent. In addition, the first biologically active agent and the second biologically active agent can be included in two different types of hydrogels. This can allow the microneedle array and drug delivery device thereof to deliver different biologically active agents at varying depths to a subject, as well as under spatial and temporal control. In addition, the microneedle array can include any number of different microneedle populations with varying lengths, varying biologically active agents, and/or varying hydrogels. For example, the microneedle array can include 3, 4, 5, 10, 20, or more different populations of microneedles with varying length, varying biologically active agents, and/or varying hydrogels.

The developed arrays may have at least one different length needle that can target different depths of tissue to deliver different therapeutics, as needed. Moreover, the developed arrays may have at least two different length needles that can target different depths of tissue to deliver therapeutics, as needed.

The array can be prepared in any suitable shape, e.g., any shape that can be 3D printed known in the art. Example shapes include, but are not limited to, a square shape, a rectangular shape, a circular shape, an oval shape, and a letter shape. In addition, the shape of the microneedle is not limited, as long as it is suitable to include a hydrogel core. Examples of microneedle shapes include, but are not limited to, a conical shape, a circular truncated cone shape, a quadrangular pyramid shape, a triangular pyramid shape, and a konide-like shape. In some embodiments, the microneedles have a triangular pyramid shape. In addition, the microneedle array can include different populations of microneedles where the different populations have different shapes.

The microneedle array described herein can combine 3D printing fabrication, multi-length arrays, core-and-shell structure, controlled hydrogel release, semi-flexible structure, and a rapid filling device and technique. In addition, the microneedle array can also carry different cells and the spatial distribution of cells, drugs, gels, and combinations thereof can be controlled.
i. Hydrogels

A variety of hydrogels may be used to facilitate controlled release of biologically active agents from their internal porous networks. The disclosed hydrogels can protect the bioactivity of an encapsulated growth factor and allow release therefrom. Hydrogels loaded with different drugs or cells can be loaded into needles on the same array, which can create precise planar spatial control of drug delivery.

Any suitable type of hydrogel for drug delivery can be used in the disclosed microneedle arrays. The hydrogel may include a polymer. For example, the hydrogel may be a network of hydrophilic polymers. In some embodiments, the polymer is a hydrophilic polymer. The polymer may be a natural or a synthetic polymer, and may include known polymers used for tissue engineering, cell culture, biosensors, implants, etc. Suitable hydrogels can include one or more of hyaluronic acid, polyethylene glycol, polypropylene glycol, polyethylene oxide, polypropylene oxide, polyglutamate, polylysine, polysialic acid, polyvinyl alcohol, polyacrylate, polymethacrylate, polyacrylamide, polymethacrylamide, polyvinyl pyrrolidone, polyoxazoline, polyiminocarbonate, polyamino acid, hydrophilic polyester, polyamide, polyurethane, polyurea, poly(ethylene glycol diacrylate), poly (l-lactic acid) (PLLA), poly(d,l-lactide-co-glycolide) (PLGA), polycaprolactone (PCL), polyglycolic acid (PGA), polyvinyl alcohol, polyacrylic acid, poly(N-isopropylacrylamide), dextran, agarose, xylan, mannan, carrageenan, alginate, gelatin, collagen, albumin, cellulose, methylcellulose, ethyl cellulose, hydroxypropylmethyl cellulose, hydroxyethyl starch, chitosan, nucleic acids, silk, derivatives thereof, co-polymers thereof, and combinations thereof. Examples of natural hydrogels include those derived from animal tissues, such as gelatin.

In some embodiments, the hydrogel includes poly(ethylene glycol diacrylate), poly (l-lactic acid) (PLLA), poly(d,l-lactide-co-glycolide) (PLGA), polycaprolactone (PCL), polyglycolic acid (PGA), polyvinyl alcohol, polyacrylic acid, poly(N-isopropylacrylamide), gelatin methacryloyl, alginate, collagen, gelatin, fibrin, hyaluronic acid, silk, blood derived materials, poly(N-isopropylacrylamide) (PNIPAm), or a combination thereof. In some embodiments, the hydrogel includes poly(ethylene glycol diacrylate), poly (l-lactic acid) (PLLA), poly(d,l-lactide-co-glycolide) (PLGA), polycaprolactone (PCL), polyglycolic acid (PGA), poly(N-isopropylacrylamide), gelatin methacryloyl, alginate, collagen, gelatin, fibrin, hyaluronic acid, or a combination thereof. In some embodiments, the hydrogel includes poly(ethylene glycol diacrylate) or alginate.

The hydrogel may include the polymer at varying amounts. For example, the hydrogel may include the polymer at about 0.1% to about 20% by (weight/volume) of the hydrogel, such as about 0.5% to about 18% by (w/v) of the hydrogel, about 1% to about 15% by (w/v) of the hydrogel, or about 1% to about 10% by (w/v) of the hydrogel. The amount of the polymer can be modulated to manipulate properties of the hydrogel and microneedle array thereof. In some embodiments, the hydrogel includes the polymer at about 0.5% to about 5% (w/v) of the hydrogel.

The hydrogel may include a plurality of pores. The pores may be interconnected to form a porous network throughout the hydrogel. Each individual pore may have a diameter of about 10 nm to about 250 μm, such as about 15 nm to about 200 μm, about 10 nm to about 150 μm, or about 20 nm to about 200 μm. Each individual pore may be at least 20 nm, at least 40 nm, at least 60 nm, at least 80 nm, at least 100 nm, at least 200 nm, at least 500 nm, at least 1 μm, or at least 5 μm. Each individual pore may be less than 250 μm, less than 240 μm, less than 230 μm, less than 220 μm, less than 210 μm, less than 200 μm, less than 190 μm, less than 180 μm, or less than 170 μm.

The hydrogel can include agents that can be useful for drug delivery applications. For example, the hydrogel can include a biologically active agent, nanoparticles, microparticles, or combinations thereof. In some embodiments, the hydrogel includes a biologically active agent. Generally, any biologically active agent which can be effectively delivered transdermally can be delivered using the microneedle arrays of the present disclosure. Examples of biologically active agents include, but are not limited to, antibacterial compounds and agents, antibiotics, small molecule drugs, large molecule drugs, proteins, vitamins, lipids, phospholipids, fatty acids, biological factors, polysaccharides, nucleic acids, growth factors, quaternary ammonium compounds, liquid crystals, peptides, chitosan, silver nitride, platelet rich plasma, blood derived materials, and combinations thereof. In some embodiments, the biologically active agent includes a nucleotide, a polynucleotide, a protein, a peptide, a polypeptide, a carbohydrate, a lipid, a small molecule drug, a cell, or a combination thereof. In some embodiments, the biologically active agent includes a peptide, a polypeptide, or a protein. In some embodiments, the biologically active agent includes a growth factor. In some embodiments, temporal release kinetics of biologically active agents from the microneedles can be controlled by changing the hydrogel composition as well as the microneedle's geometry.

As mentioned above, the hydrogel can also include a plurality of nanoparticles. Nanoparticles can have an average diameter of about 1 nm to about 900 nm. Example nanoparticles can be of any shape and can be made from materials including, but not limited to, metal, metal oxides, bioglasses, radiopaque agents, bioceramics, ceramics, oxygen generating materials, graphene, graphene oxide, carbon derived materials, and combinations thereof. The nanoparticles, themselves, can also include a biologically active agent as described herein.
ii. Methods of Making

Also disclosed herein are methods of making the microneedle arrays. The described microneedle arrays may utilize a 3D-printed core-and-shell approach wherein a hard resin outer shell is printed, with an end of the needle left open. Accordingly, the method can include printing a microneedle array, such as by 3D printing techniques. 3D printing allows for simple and rapid design changes, including array shape and area, needle shape and density, and needle length to accommodate individual patient needs. The description of the microneedle array above can be applied to the methods of making as well.

The method can further include adding a prepolymer to the channel of each microneedle. The prepolymer can be added to the channel under vacuum. The prepolymer is a material that forms into a hydrogel after, e.g., crosslinking the prepolymer. Accordingly, the method can also include crosslinking the prepolymer to provide a hydrogel in the channel of each microneedle. Crosslinking of the hydrogel can be achieved via ionic crosslinking, covalent crosslinking, or a combination thereof. In addition, the prepolymer can be crosslinked via enzymatic crosslinking, thermal crosslinking, photo-crosslinking, or a combination thereof. In some embodiments, the printed microneedle array, with the prepolymer positioned in the core of each microneedle, is placed into a crosslinking source, where once placed into the crosslinking source, the prepolymer is crosslinked to provide the hydrogel. In some embodiments, the crosslinking source is an agarose gel that includes a divalent ion, such as CaCl₂.

A loading device (e.g., pipette attachment) may be used to fill the hollow core of each needle in the array with a drug-laden hydrogel prepolymer. In some embodiments, the pipette attachment is used to fill the hollow core of each needle in the array with a drug-laden hydrogel prepolymer simultaneously (or near simultaneously). In addition, the loading device can uniformly fill the hollow core of each microneedle. The hydrogel may then be crosslinked before use of the microneedle array. The loading device can include a base having a first end and a second end. The loading device can further include a plurality of outlets coupled to the first end of the base that align in number and arrangement with the plurality of microneedles, e.g., that are being filled with the prepolymer. The loading device can also include an outlet coupled to the second end of the base in fluid communication with the plurality of outlets. The loading device can be configured to align with any type of plurality of microneedles (e.g., varying shape, hydrogel, length, etc.) that would require filling of their cores. The method can also include washing the printed microneedle array with a base solution prior to addition of the prepolymer.

### 3. Methods of Treating Disorders

Also disclosed herein are methods of treating a disorder. The method can include treating a disorder in a subject in need thereof, the method including contacting an area of the subject's skin with the disclosed drug delivery device, wherein the biologically active agent is transdermally delivered to the subject. The method can include delivering at least 2, at least 3, at least 4, at least 5, or more of different biologically active agents. Examples of disorders include, but are not limited to, burns, diabetic ulcers, chronic wounds, muscle abnormalities, melanoma, cancer immunotherapy, and keloids. The description of the microneedle array and drug delivery device thereof above can be applied to the methods of treating as well.

In non-limiting examples, the microneedle array and drug delivery device thereof described herein can be used for a wide variety of biomedical applications such as treatment of burns or chronic wounds, muscle injuries. They can also be used for cell and stem cells delivery.

The disclosed invention has multiple aspects, illustrated by the following non-limiting examples.

### 4. EXAMPLES

### Example 1

### Materials & Methods

Materials. VeroClear RGD810 and TangoBlack FLX973 resins were obtained from Stratasys (MN, USA) and used for 3D printing of MNA dressings. Materials and reagents including NaOH, alginic acid sodium salt, bovine serum albumin (BSA), Irgacure 2959, calcium chloride (CaCl₂), agarose, ultra- low gelling agarose, poly(ethylene glycol diacrylate) (PEGDA), BSA fluorescein isothiocyanate conjugate (BSA-FITC), and HUVEC culture media were purchased from Sigma-Aldrich (MO, USA). Other reagents including Human VEGF 165, Dulbecco's phosphate-buffered saline (DPBS), fetal bovine serum (FBS), trypsin-EDTA, Rhodamine B (Rh-B) and Micro BCA kit were obtained from ThermoFisher Scientific (MA, USA).

Fabrication of MNAs. The MNAs were designed in SolidWorks (Dassault Systems, France). All arrays were printed using a material jetting (MJ) Stratasys Objet 500 Connex3 3D printer (MN, USA). The needles and the portion of backing directly below them were printed out of VeroClear resin, while the ribs between the islands were printed using TangoBlack, simulated rubber material to make the dressing flexible. Support material from the printer was primarily mechanically removed, with the remainder of residues being washed off through soaking in 3% (w/v) NaOH solution suspended in an ultrasonic bath. Prior to being used for the experiments, MNAs were thoroughly rinsed with an ethanol solution (70% v/v) for disinfection and subsequently in DPBS. For the experiments using alginate, loading was performed while MNAs were fully inserted into a 3% (w/v) agarose gel containing 2% (w/v) CaCl₂ to crosslink, and in experiments with PEGDA, 30 sec of UV exposure was employed for crosslinking the loaded solution containing 1% (w/v) Irgacure 2959 as the photoinitiator. Arrays containing different needle lengths were also similarly printed.

Design and Fabrication of Loading Mechanism. The multi-needle filling attachment was designed in SolidWorks with conical channels to mimic the structure of a pipette tip and to facilitate the movement of fluids down and out of the reservoir. It was fabricated using VeroClear with an approach similar to the MNAs. The receiving end of the attachment was designed to fit a P200 micropipette. Following fabrication, the attachment was affixed to a pipette and loaded with the precursor solution, including a 1% (w/v) alginate solution, which was then distributed to each target needle. The solutions were then crosslinked as described before.

Mechanical Characterization of the Engineered MNAs. Mechanical properties of MNAs against compressive mechanical loads were measured using a CellScale Univert Mechanical tester (ON, Canada). The arrays were affixed to the lower jaw of the tester, the upper jaw was lowered until contact was made, and the mechanical load was applied. A maximum of 200 N compressive force was applied as the jaw was moved at a rate of 0.092 mm/s.

The penetration capability of MNAs with different compositions was also evaluated using the CellScale Univert Mechanical tester. The penetration test was performed using three categories of MNAs: all VeroClear arrays, mixed material semi-flexible arrays, and arrays fully fabricated on TangoBlack backing all with 3 mm long needles. A 3×3 cm square of pig skin was glued to the lower jaw of the tester and MNAs were glued to the top jaw. The upper jaw was lowered until the tips of the needles were in contact with the pig skin. The compression force was applied as the jaw was moved at a rate of 0.27 mm/s until the needles were fully penetrated into the skin sample. The samples rested for 1 min before removal at the same rate to measure pull out force.

Hematoxylin and Eosin Staining (H&E). Samples of pig skin penetrated with needles were fixed in 10% formalin for 48 h, followed by immersion in 70% ethanol for 24 hrs. The needles were then retracted, and the samples were cut and embedded in paraffin blocks. The paraffin-embedded skin samples were sectioned into 6 μm thick slices to expose the cross section. Finally, the sections were deparaffinized and stained with H&E. The samples were then inspected using a Zeiss microscope and images were obtained.

Assessment of Release Kinetics. The potential of the system for the delivery of different drugs encapsulated in various hydrogels at different concentrations was evaluated by assessing the release kinetics of BSA and Rh-B. To perform the release experiments, either BSA or Rh-B was mixed with a hydrogel precursor solution and then loaded into MNAs with 8 needles and then crosslinked. For BSA release experiments, 3 mm-long needles were filled either with 1% (w/v) alginate or 23% (w/v) PEGDA. The alginate solution was prepared with a stock solution of BSA at a concentration of 2000 μg/mL, while the PEGDA solution was loaded with 1250 μg/mL of BSA. MNAs were placed in a 24-well plate. Each well was filled with 0.5 mL of DPBS and sampling at each time point was performed by removing the entire solution and storing it in a microvial and replacing the sampled volume with fresh DPBS. The BSA content at each time point was assessed with a Micro BCA™ Protein Assay Kit (Thermo Scientific™) following the manufacturers recommended protocol using a BioTek Cytation 5 plate reader (VT, USA). To compensate for the material interference in the absorbance results, a control group of similar MNAs filled with the same hydrogel, but not carrying any additional molecule, was used. These results were subtracted from the loaded hydrogel release values.

In Rh-B release experiments, solutions of 1, 2, and 3% (w/v) sodium alginate were mixed with Rh-B such that complete release would result in a final concentration of 20 μg/mL in 3 mm-long needles. The same solution was loaded into the 2 mm-long needles. The MNAs were filled and crosslinked with the same procedures described before. During sampling, only 150 μL of the solution was removed and replaced at each time point. The samples' absorbance values at 560 nm wavelength were read using the BioTek Cytation 5 plate reader. All of the release experiments were performed at least in triplicate.

Evaluation of Dual-Drug Release Capability. For the dual release, MNAs with 4.2 mm long and 2 mm short needles were separately loaded with a 1% (m/v) alginate solution in DPBS containing ˜46 μg/mL of Rh-B, ˜1010 μg/mL of BSA-FITC, or no added analyte. Four combinations of analyte-loaded alginate MNAs were tested: (1) long needles loaded with Rh-B and short needles loaded with BSA-FITC (Rh-B/FITC), (2) long needles loaded with Rh-B and short needles blank (Rh-B/Blank), (3) long needles blank and short needles loaded with BSA-FITC (Blank/FITC), and (4) both lengths blank (Blank/Blank). The gels were crosslinked as previously mentioned and ran in quintuplicate. The needles were inserted into a two-gel skin model that include a 2 mm thick 3% (m/v) low-gelling agarose cast on top of a 3 mm thick 10% (m/v) porcine gelatin in 12 well plates. Both solutions were prepared in PBS. A two-gel model was selected for this experiment both to simulate multiple distinct layers of skin and to allow for easy separation of the two layers. Ultra low-gelling agarose was selected for the second gel because it is stiff, does not interact with gelatin, and could be melted to take samples. The stiffer agarose gel was cast as the top layer to better mimic the mechanical barrier that the epithelium presents. The initial analyte concentrations loaded into the needles were designed for final concentrations of 20 μg/mL of FITC-BSA in the top agarose layer and 2 μg/mL of Rh-B in the bottom gelatin layer. After 16 h, the needles were removed from the two-gel skin. The gels were separated and collected in different containers. The collected gels were melted and the amount of Rh-B and BSA-FITC was quantified using a BioTek Cytation 5 plate reader at an excitation/emission of 546/568 and 495/520 nm, respectively.

Scanning Electron Microscopy (SEM). The microstructure of the alginate hydrogel and gel-filled MNAs were analyzed using SEM. To visualize the porous nature of the alginate, crosslinked alginate hydrogel and gel-filled MNA were immediately frozen in liquid nitrogen. The frozen samples were then lyophilized for 2 days using a FreeZone® Benchtop Freeze Dryer (Labconco®). The crosslinked alginate hydrogel was broken to reveal the internal microstructure. The cross section of the alginate and the filled MNAs were sputtered with palladium using a sputter coater (Cressington 106 Auto Sputter Coater). The images were captured using a scanning electron microscope (FEI Quanta 200 Environmental) at 10 kV and under a high vacuum.

In Vitro Scratch Assay. Human umbilical vein endothelial cells (HUVECs) (Sigma-Aldrich) were cultured in EGM™ Endothelial Cell Growth Medium BulletKit™ (Lonza) complete medium. At their fifth passage, the HUVECs were detached using trypsin-EDTA (0.1% w/v), resuspended in the growth medium, and seeded at a concentration of 50,000 cells per well into a 12-well tissue culture plate that was coated with a thin layer of Geltrex® hESC-Qualified, Ready-To-Use, Reduced Growth Factor Basement Membrane Matrix (Gibco®). The Geltrex® layer was formed by diluting by a factor of 2 in the basal endothelial medium and seeding in empty wells. The Geltrex® was incubated at 37° C. for 1 h, and then any liquid Geltrex® was aspirated. The growth medium was changed every 2 days until the cells became confluent. The cells were starved 12 h before the induction of a scratch by changing the medium to basal medium supplemented with 2% (v/v) fetal bovine serum (Lonza).

A scratch was created in the confluent HUVECs by lightly dragging a P200 pipette tip across the bottom of the well. The medium was changed post scratch to remove any debris and cell fragments from the creation of the scratch. Groups were randomly assigned before determining the initial time point of the scratch. Three groups were assigned and tested (n=4): 1) 1% alginate in MNAs without VEGF (MNA−VEGF), 2) 1% alginate in MNAs with VEGF at a concentration of ˜6.250 μg/mL (MNA+VEGF), and 3) positive control with FBS-basal medium supplemented with ˜50 ng/mL of human VEGF (Invitrogen). The MNAs were placed in cell culture wells that were prefilled with 2 mL of FBS-basal medium. The concentration of VEGF in the MNA+VEGF group was calculated so that if total VEGF release occurred, the concentration of VEGF in the well would match the concentration of the positive control (50 ng/mL). Images of the scratches were captured before treatment (0 h) and at various time points after treatment (1 h, 4 h, and 8 h). The area of scratch closure for each time point was calculated using ImageJ (NIH, Bethesda, Md.) and compared for relative area change to 0 h.

Statistical analysis. Statistical analysis was performed using GraphPad Prism 8. Values are reported as mean±standard deviation. All tests were run at least in triplicate. ANOVA was performed for grouped analysis, followed by a Tukey's post-comparison post-test with a p-value of less than or equal to 0.05. p-values of less than 0.05 were considered statistically significant. A p-value less than 0.05 in indicated by *, a p-value less than 0.01 is indicated by **, a p-value less than 0.001 is indicated ***, and a p-value less than 0.0001 is indicated ****.

### Example 2

### Microneedle Array Fabrication & Characterization

In addition to the potential for transdermal delivery of drugs for systemic administration from MNAs, there are many skin injuries and disorders that could benefit from transdermal delivery of therapeutics. For example, in chronic wounds, where healing is impaired, it has been shown that the delivery of drugs and biological factors can boost physiological healing processes, but their efficacy is dependent on the delivery method. Since, upon injury, the skin barrier is breached, it has been believed that the topical delivery of drugs will allow their proper penetration and distribution. However, the formed eschar, a crust of necrotic tissue that dries and hardens post-injury, combined with exudate flow rich with various enzymes can lower the bioavailability of topically delivered compounds to the target cells. Additionally, spatiotemporal control has been identified as an important characteristic of effective bioactive factor delivery systems. Therefore, it is hypothesized that controlling the spatiotemporal distribution of various biological factors and drugs will improve the healing rate of chronic wounds and other skin conditions.

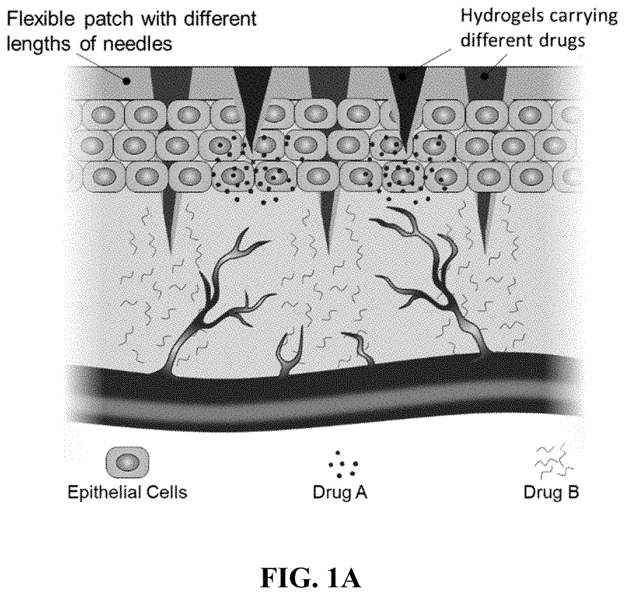

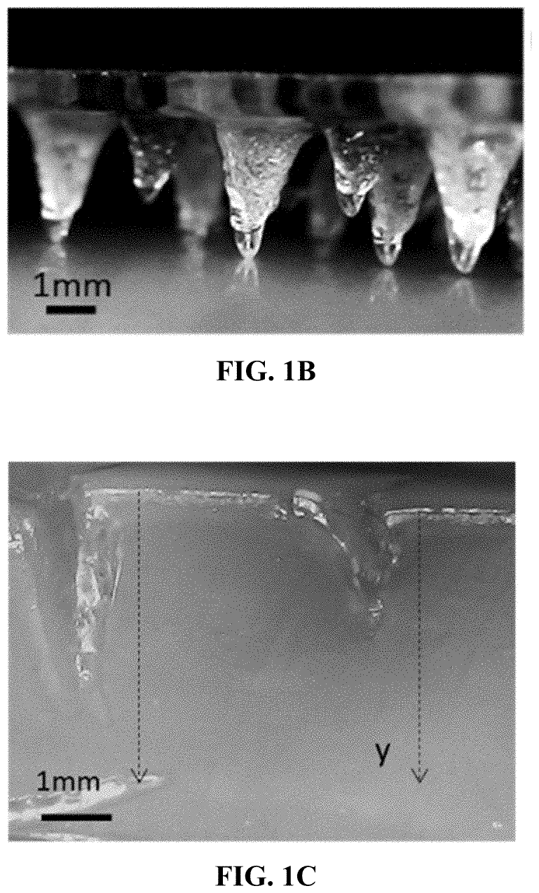

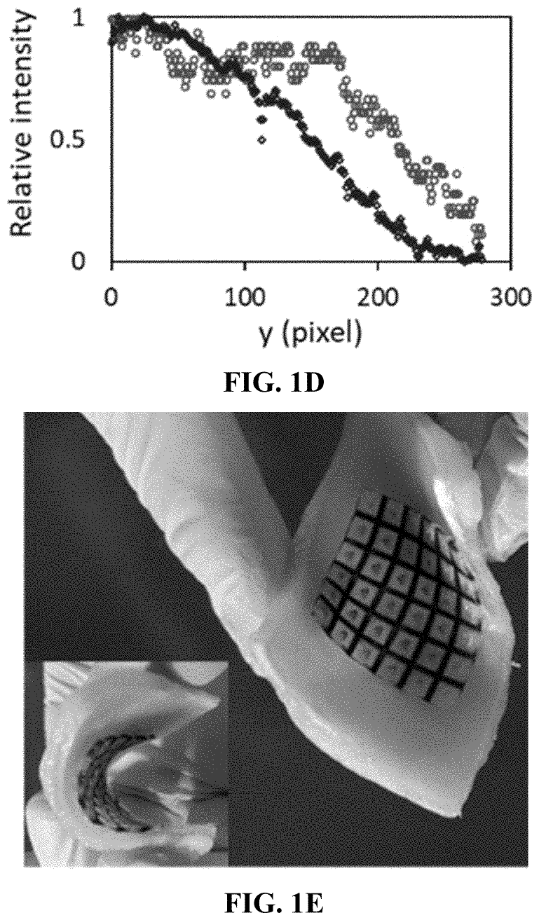

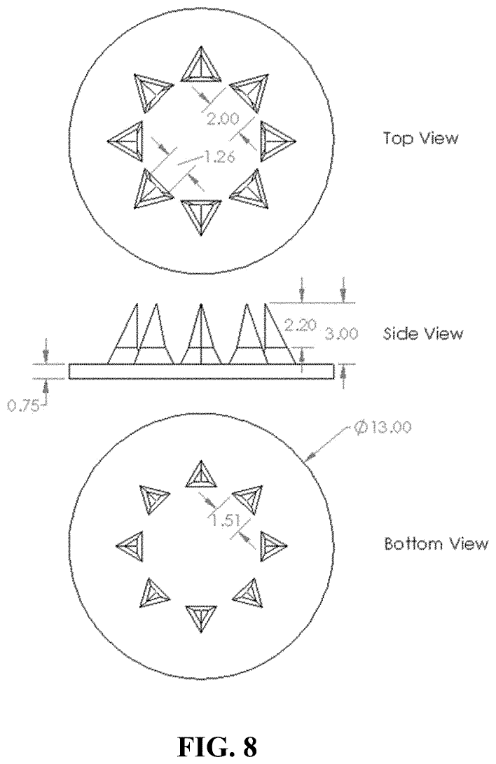

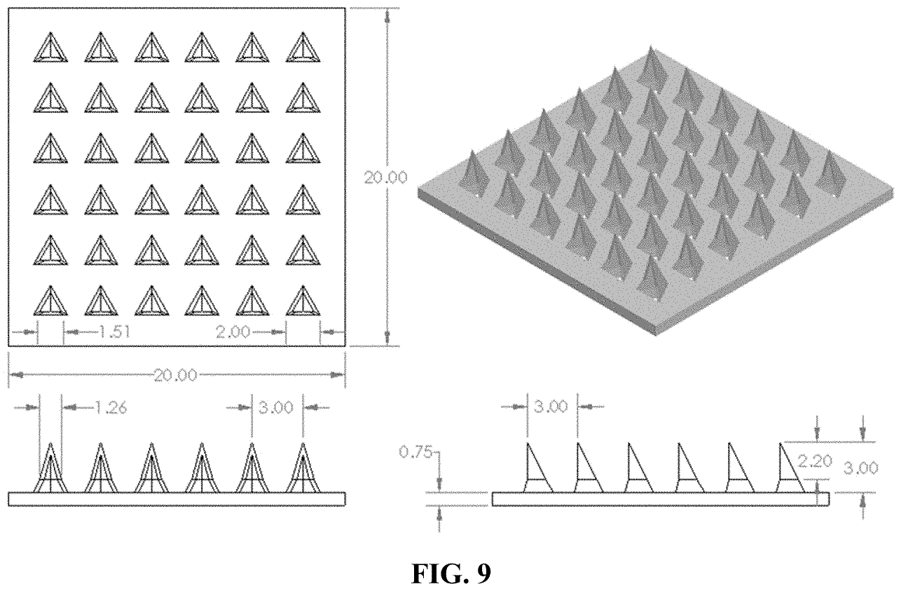

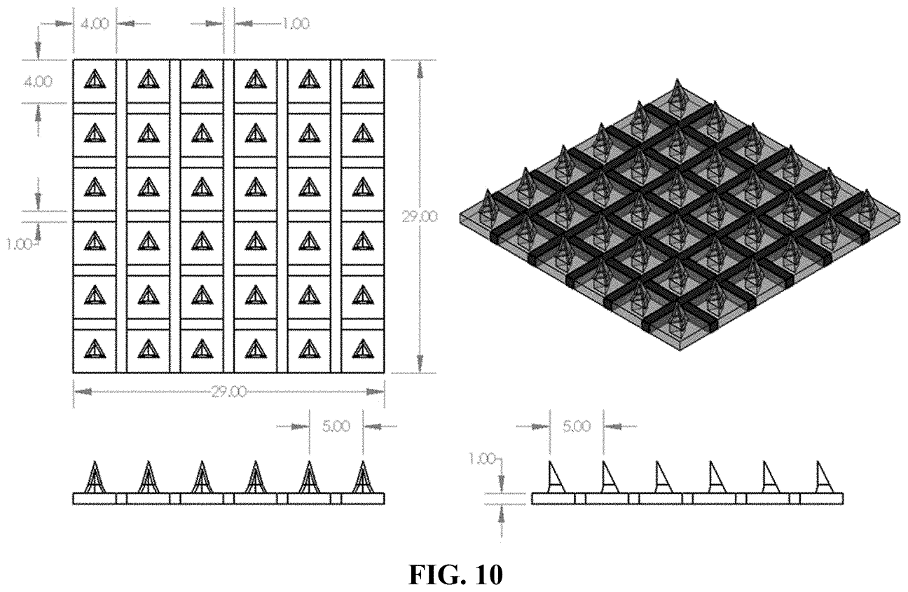

A strategy was developed that enabled the spatiotemporal control of delivered drugs to different layers of the skin. The platform is composed of MNAs with different needle lengths, in which each individual needle can be loaded independently. A schematic illustrating the developed platform is shown in FIG. 1A, demonstrating that by encapsulating drugs in different hydrogels integrated within these needles, the release kinetics of each drug can be customized. Furthermore, it is illustrated how needles of different lengths enable a spatially controlled distribution of drugs across the depth of the skin tissue. For ease of in-lab manipulation in this proof of concept study, 2 mm and 3 mm needles were utilized to demonstrate the trends in release depth and kinetics. These needle lengths have the potential to be painful during application; however, these values are tunable and could be shortened for in vivo use. Depending on the location on the body, the human epidermis can vary in thickness from 0.1 mm to 1.5 mm and the dermis can range from less than 1 mm to 5 mm, so the needle lengths can be adjusted to meet the needs of the skin at the administration site. The MNAs could also be coated with an anesthetic as needed. Additionally, there are a number of applications where patients have experienced nerve destruction, including severe burns and neuropathic diabetic chronic wounds, and could benefit from the controlled intradermal drug delivery without risk of pain. Animal work has also been done with needles of a similar length and shown success at improving wound healing. In the case of a chronic wound application, the shorter needles would target the epidermis while longer needles could penetrate into the dermis and release their payload into these deeper layers. As an example, longer needles can be loaded with VEGF-containing hydrogels to deliver these biochemical cues to the existing vasculature in the dermis. VEGF, a well-established pro-angiogenic factor can enhance angiogenesis in the wound bed, resulting in enhanced perfusion of nutrients and oxygen while allowing the infiltration of immune and stem cells to the wound bed, and ultimately facilitate tissue repair that had been hindered by lack of VEGF. Antibiotics, on the other hand, could be loaded into the shorter needles and be delivered specifically to the necrotic tissue typically colonized with pathogens. This would lead to the elimination of infection without interrupting the healing process due to the antibiotic adverse effects. A representative picture of a typical dressing with two different needle lengths, in which each group carries a different dye, is shown in FIG. 1B. Upon insertion into an agarose gel, the hydrogels embedded inside the needles began to release their cargo. The spatial distribution of different dyes within the agarose gel is demonstrated (FIG. 1C). Longer needles can increase the bioavailability of drugs in the deeper layers of the skin-mimicking gel, while the payload in the shorter needles is concentrated closer to the surface (FIG. 1D). The final MNA design was composed of rigid resin needles and islands held together with a more compliant, rubber-like material, providing both stability for insertion and flexibility to conform to topographical skin surfaces (FIG. 1E). Other designs can be seen in FIG. 8, FIG. 9, and FIG. 10.

MNAs have been fabricated using a wide variety of materials and different strategies such as micromolding, laser ablation, drawing, and photolithography. While these microfabrication approaches offer good structural resolution, they rely on expensive facilities, their flexibility is limited, and/or they have a low throughput. Also, while they can be used to fabricate small-scale structures, typically fabrication of these structures with multiple heights is not feasible. Recent advancements in 3D printing has improved resolution, reduced costs, and increased throughput. Also, inherent to the 3D printing technique is the ability to fabricate complex structures, such as multi-height MNAs.

A limitation of using hydrogels to fabricate MNAs is their poor mechanical properties for forming high aspect ratio structures capable of penetrating the skin. To overcome this challenge, a bilayered MNA was developed in which the exterior was formed from a rigid biocompatible resin and the interior was filled with hydrogels carrying biologically relevant agents. In this example, alginate and PEGDA were tested as two examples of ionically- and covalently-crosslinkable hydrogels, respectively.

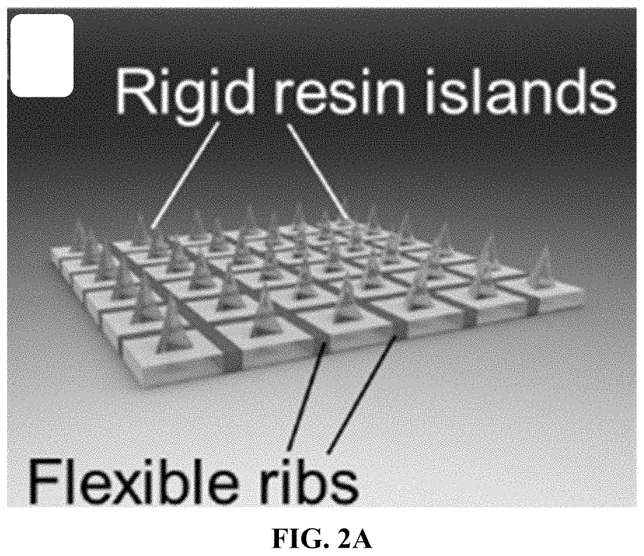

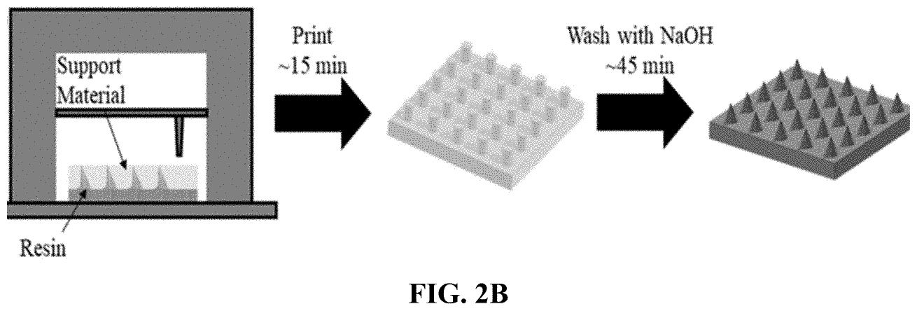

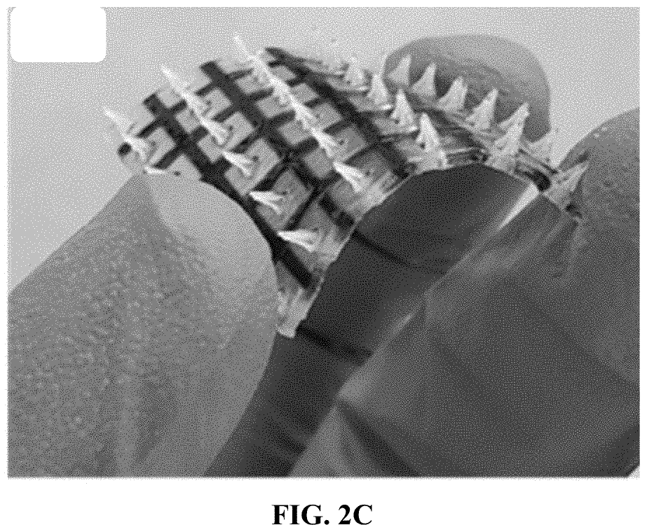

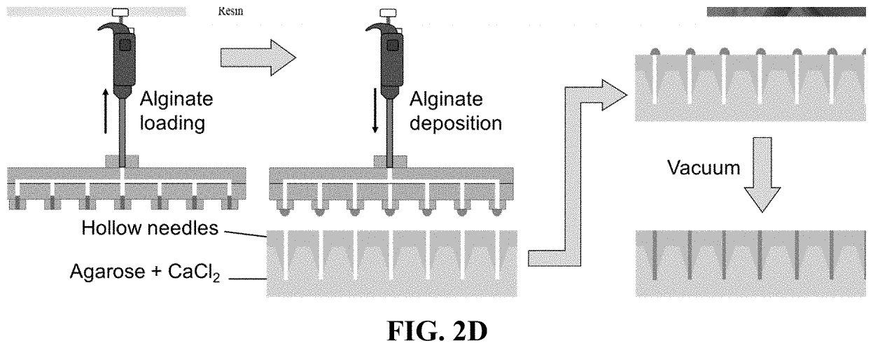

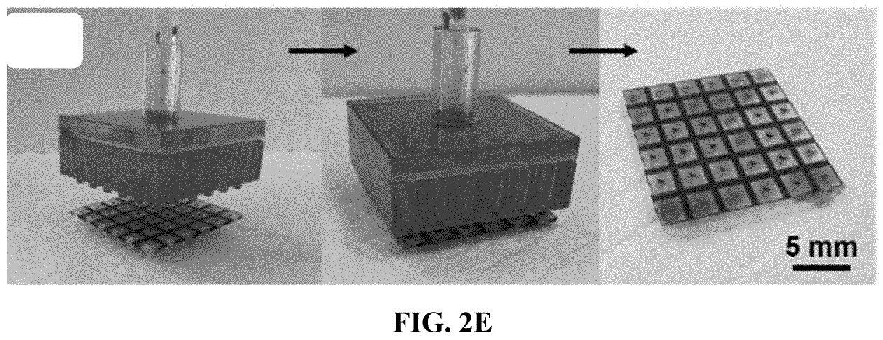

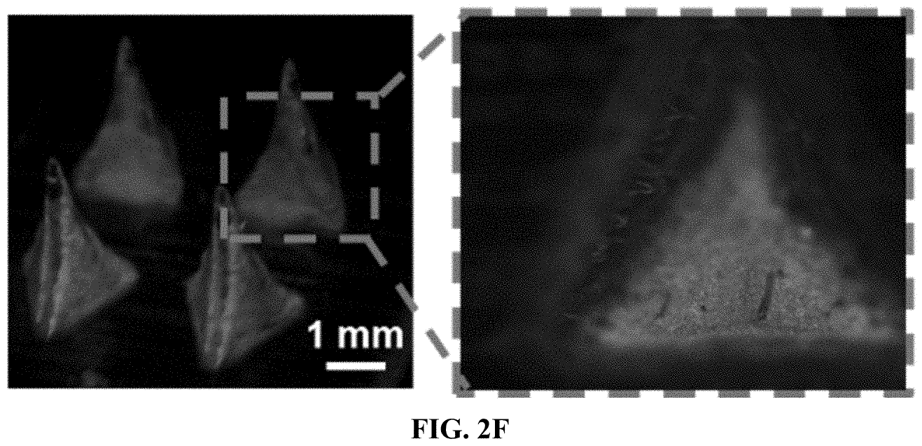

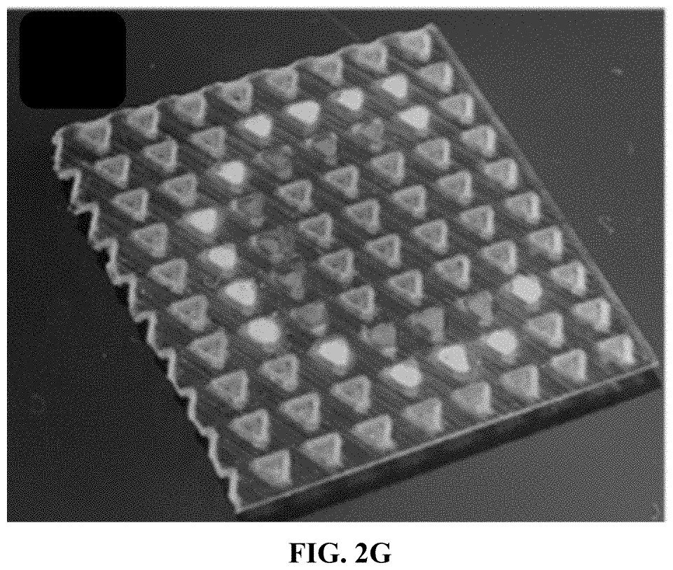

The designed MNAs (FIG. 2A) were printed using a material jetting (MJ) 3D printer and then the support material was removed using a NaOH solution to expose the resin construct underneath (FIG. 2B). The printer that was used can print objects with a combination of materials. This capability allows for the fabrication of MNAs with a tunable degree of flexibility using rigid needles connected by flexible and rubber-like ribs (FIG. 2C). The semi-flexible dressing maintained appropriate needle rigidity and orientation during insertion while providing flexibility to conform to curved body surfaces or uneven wound topography (FIG. 2C).

To facilitate and enhance the reproducibility and controllability of the filling process, we created a loading mechanism that can fill the entire MNA at once or a designated section, a concept that is easy to scale up and automate. During the needle loading process (FIG. 2D), the array was inserted into an agarose gel containing CaCl₂. The attachment was loaded with an alginate prepolymer through normal pipette operation, with fluid flowing into all of the channels. The prepolymer could then be precisely deposited over each needle on an array. The array was placed in a vacuum chamber to force the prepolymer to flow into the needles, where it contacted the agarose and was crosslinked by the calcium ions contained therein. Additional CaCl₂ solution was pipetted over the backs of the needles to ensure complete crosslinking.

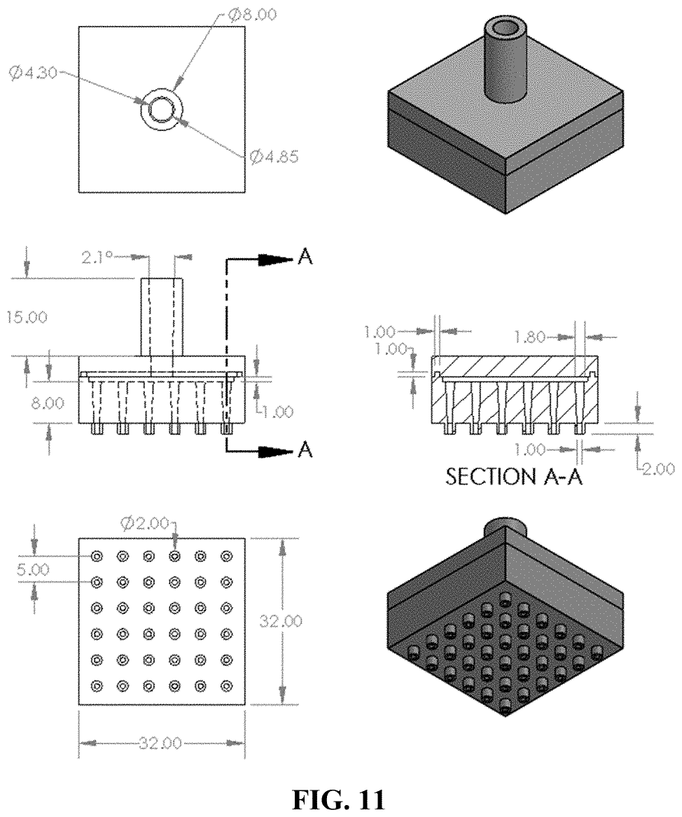

This mechanism is designed to fit on a P200 micropipette and has 36 conical channels with dimensions to match the semi-flexible MNAs. It was tested with a 1% alginate solution mixed with Rh-B for better visualization. The prepolymer was easily pulled into the attachment channels and deposited over the needle openings on the MNA (FIG. 2E, see also FIG. 11).

To show the space occupied by the hydrogel, resins in the MNAs were stained with Rh-B and then filled with alginate hydrogel containing Fluorescein isothiocyanate (FITC). The MNAs were imaged using a fluorescent microscope (FIG. 2F). Each needle includes a hollow triangular pyramid with one side open and exposed to allow the contained hydrogel to interface with the tissue environment which allows diffusion of the therapeutic molecules. The large surface area facilitates controlled release through diffusion, while the structure of the needle keeps the hydrogel contained, and therefore, removable. Unlike an injection delivery method, a removable patch allows for easy withdrawal in the case of any adverse reaction to the incorporated compounds, or in response to a change in the wound environment. One important benefit of the proposed fabrication approach is the ability to control over the spatial distribution of the loaded therapeutics.

In this case, by using predesigned loading systems the planar distribution of drugs within the patch can be controlled. FIG. 2G shows a typical patch in which two “C-shaped” patterns of needles were formed with blue and red colored gels within an array filled with transparent gels.

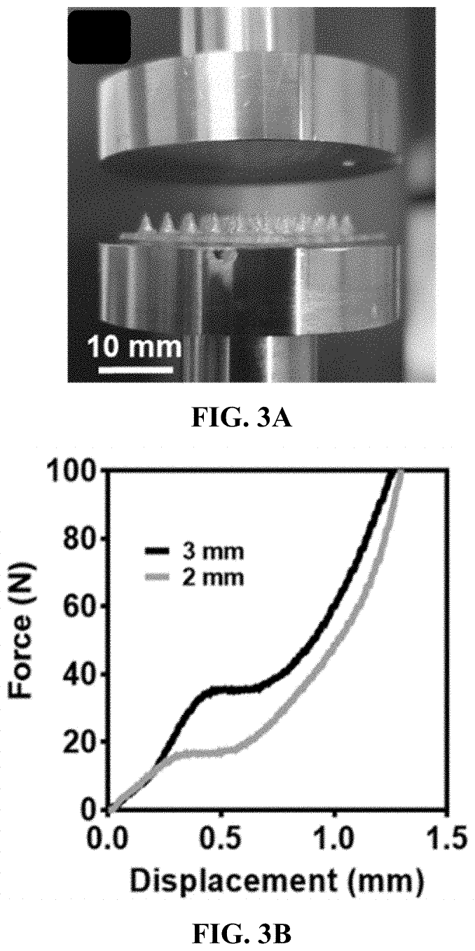

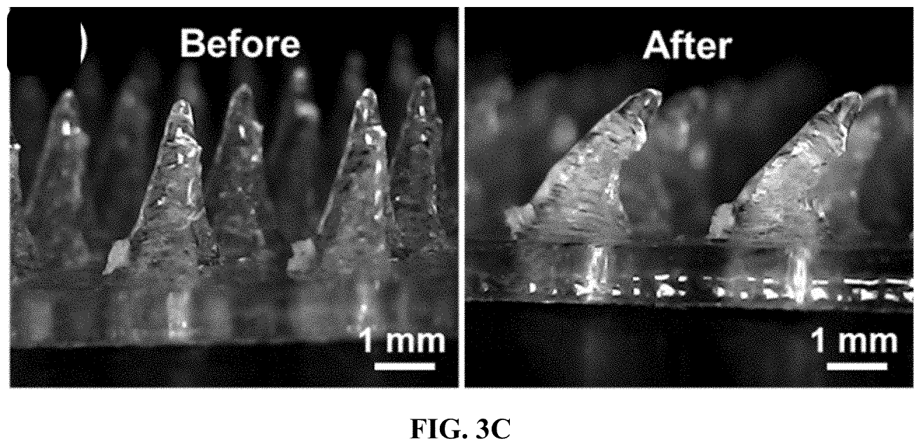

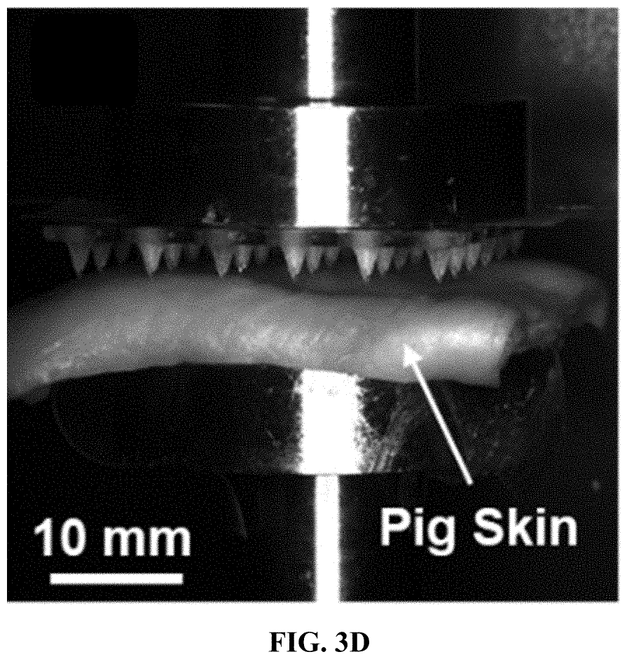

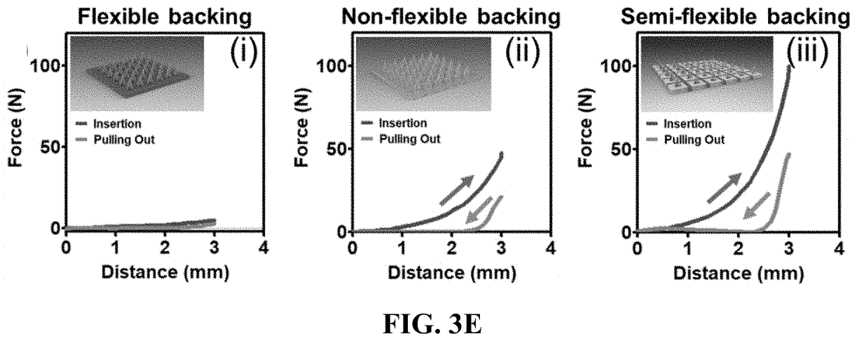

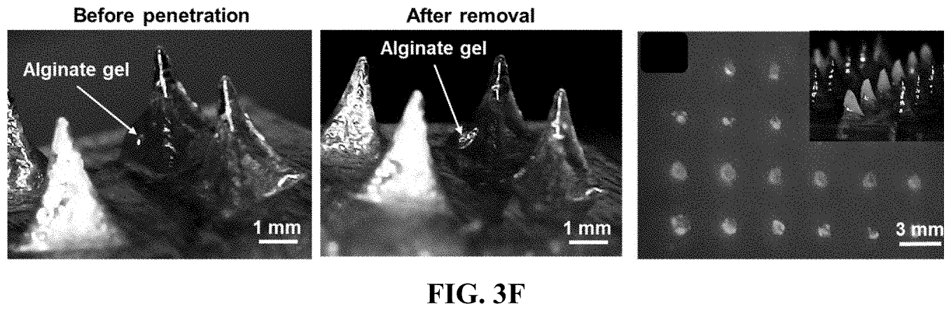

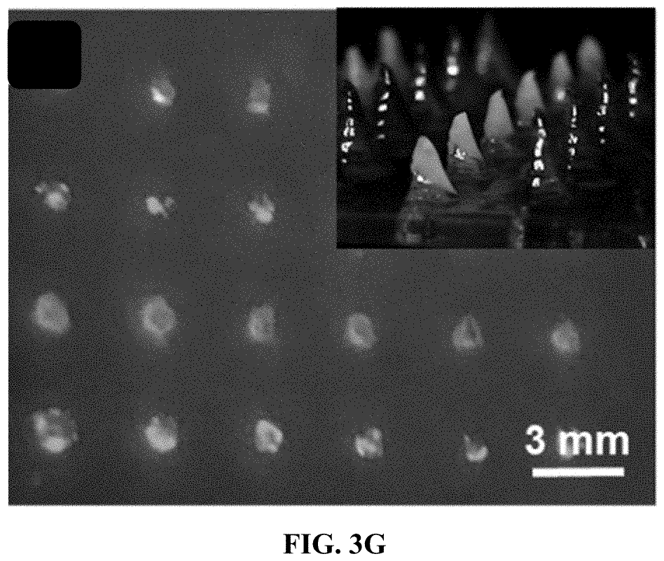

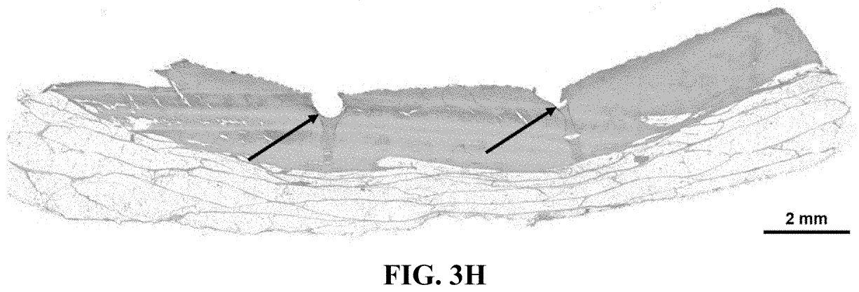

The mechanical properties of the MNAs were evaluated to ensure they were mechanically robust enough that they did not sustain any damage upon insertion into a wound (FIGS. 3A-H). Compressive force was applied to the needles up to and past their failure point, up to 200 N (FIG. 3A). The 2 mm-long MNAs failed at approximately 12% strain of jaw displacement, while the larger 3 mm-long failed at about 16% strain. This failure is characterized by a plateau in the force-displacement curve where the applied force is not increasing but the displacement of the upper jaw continues (FIG. 3B). MNAs failed via bending rather than being shattered, crushed, or snapped off of the substrate (FIG. 3C). It is important that if the needles fail due to excessive force, they bend rather than break off into the wound bed, which could aggravate the local inflammation. Overall, compression testing was performed to a much greater maximum force than it actually takes to insert these needles into the skin.

Next, insertion testing was performed using a pig skin model (FIG. 3D). The tests were conducted with non-flexible, flexible, and semi-flexible arrays. Non-flexible arrays were entirely fabricated from the rigid resin, while in the fully flexible design, the backing was entirely made of a rubber-like material. In the semi-flexible design, however, a single needle and its backing within the array were separated by narrow ribs of rubber-like material. As shown in FIG. 3E, the fully flexible arrays could not penetrate the pig skin. The low increase in force in the curve shown in FIG. 3E resulted from the needles bending out of plane due to deformation of the backing. As a result, the array was not stiff enough to penetrate the pig skin. The non-flexible arrays functioned as intended with the rigid backing holding the needles in place and not deforming as they successfully penetrated the pig skin. However, rigid backing cannot have a conformal contact with skin or a wound surface. The semi-flexible arrays followed a very similar force trace, suggesting that the incorporation of flexible ribs makes them conformal to the skin without affecting their ease of penetration.

The gel-filled MNAs were inspected before and after penetration into pig skin to ensure that the hydrogel would not be pulled out of the needles during removal from a wound. The images from a typical MNA, filled with a colored gel for better visualization, before and after insertion into pig skin are shown in FIG. 3F. It can be seen that the gel remained intact during needle insertion and after retraction. It is not expected that the alginate will dissociate rapidly in the wound bed as the rate of ion exchange is typically slow.

As previously discussed, the spatial distribution of delivered drugs could be controlled within the engineered patch. For example, different rows of needles were filled with gels containing either Rh-B (red) or FITC (green). The needles were then inserted into pig skin and left on the skin for several minutes. Upon needle removal, the spatial distribution of colors within the pig skin can be seen (FIG. 3G). To verify that the MNA was capable of penetrating both the epidermis and dermis, an MNA was inserted into a sample of pig skin and H&E staining was performed (FIG. 3H). The holes left by the needles can be clearly seen passing through the epidermis and into the dermis of the sample.

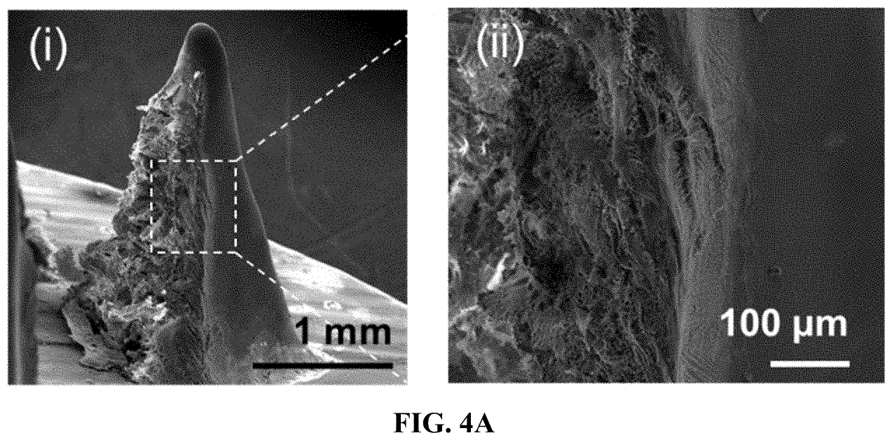

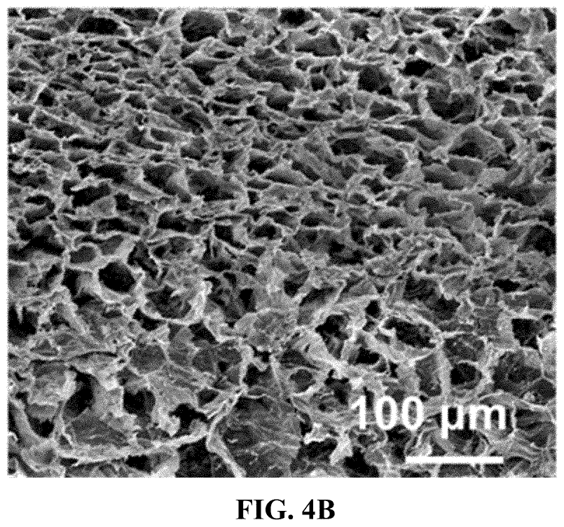

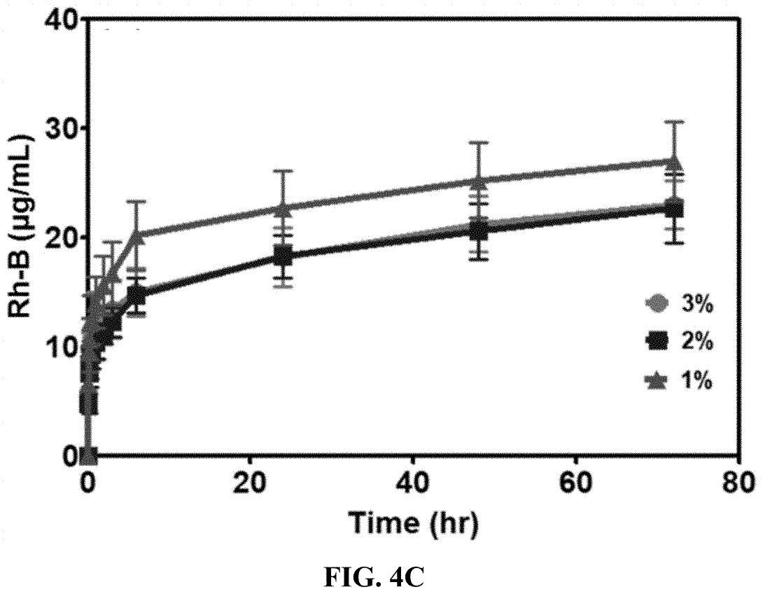

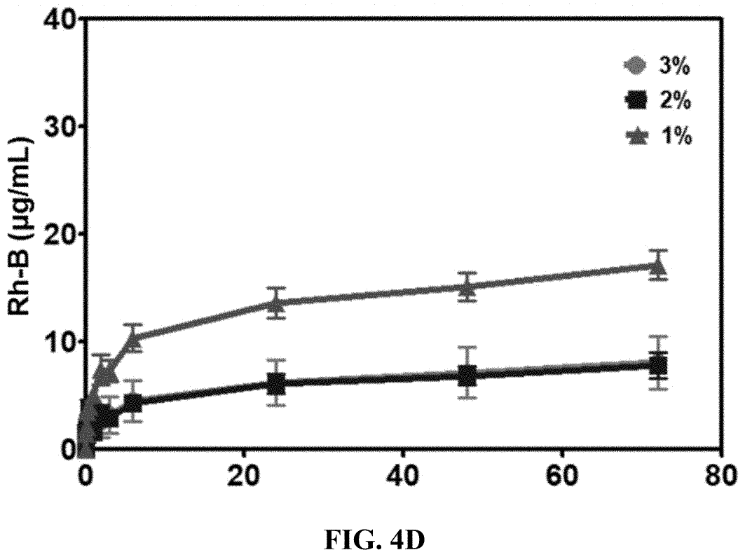

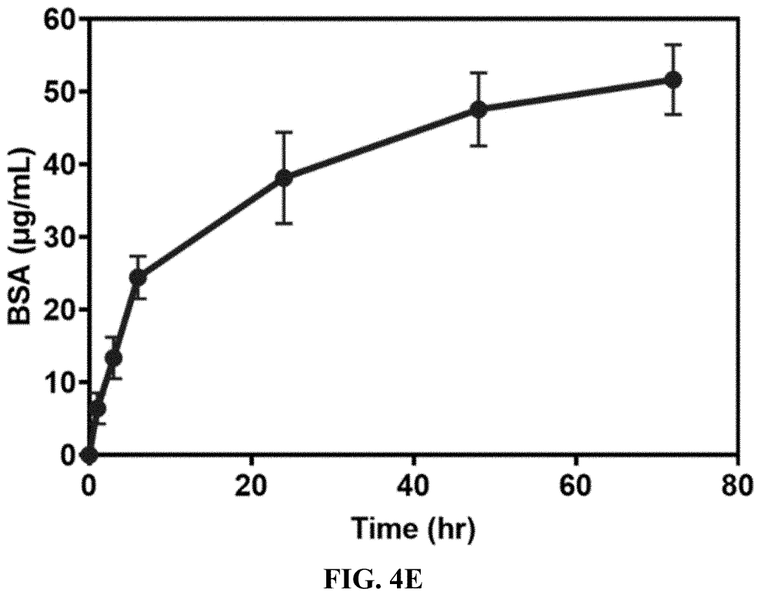

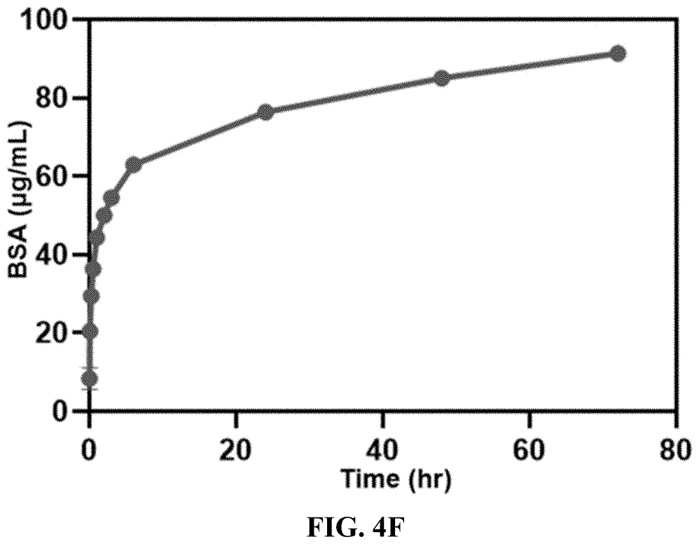

The hydrogel embedded within the MNAs was examined by SEM (FIG. 4A). To capture the internal microstructure, needles were overfilled with 1% alginate, lyophilized and the gels were broken to expose their cross section. The image also shows the gel-resin interface, highlighting the adhesion of the gel to the resin. This adhesion is crucial to ensure that when MNAs are removed from the skin, the gel remains within the needles rather than being pulled out. Higher magnification of the internal structure demonstrated the porous nature of the alginate hydrogel formed inside the needles (FIG. 4B). The porosity of hydrogels is integral to their ability to contain and deliver molecules and the pore size can affect the release kinetics. Alginate crosslinked into a network of connected pores of similar size can essentially function like a sponge to store and release the loaded biologics.

Confinement of the gel within the rigid needle structure enables easy removal of the MNA and provides the opportunity to replace it as needed. This could be necessary for transdermal delivery of therapeutics in case of an allergic or immunogenic reaction from the patient. This would also be valuable for wound treatment, because in this dynamic environment, drugs or growth factors that improve healing at one stage of healing may be useless or harmful during a different stage. The platform developed addresses these rapidly changing conditions since it can be quickly and easily replaced with another MNA loaded with new therapeutics that are most relevant at a given phase of the wound healing process. Additionally, it is clinically recommended to replace dressings every two to three days to prevent infection. This time frame was used as a basis for determining the desired period of release from the hydrogels cores. A number of release studies were conducted to determine delivery kinetics under several different conditions. The effect of hydrogel composition, molecular size of the encapsulated drugs, and needle geometry on the release kinetics was evaluated.

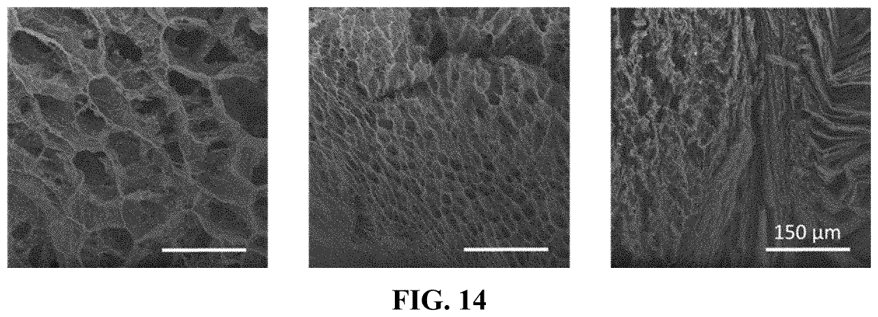

Rh-B was considered as a low molecular weight drug model, and its release from alginate embedded in the MNAs was assessed using various needle geometries and alginate compositions (FIG. 4C and FIG. 4D). First, 3 mm-long needles were filled with 1, 2, and 3% (w/v) alginate containing Rh-B to explore the effect of concentration (FIG. 4C). The release rate was higher initially and decreased after 6 h. These results suggested that increasing the alginate concentration, could decrease the release rate of Rh-B. A similar experiment was performed with 2 mm-long needles containing the same alginate gels (FIG. 4C and FIG. 4D). The release profile of Rh-B from the 2 mm-long MNAs was overall similar to the longer needles. However, the total amount of released Rh-B decreased compared to experiments with 3 mm-long needles, which was attributed to the decreased available volume of the hydrogel in the smaller needles. In both experiments, the 1% alginate released more than the 2% and 3% alginate groups, demonstrating the ability to tailor the release kinetics with hydrogel composition and pore size. Representative SEM images showing the varied pore size of 1%, 2%, and 3% alginate are presented in FIG. 14.

To test the versatility of the platform, the delivery of BSA, used as a model protein, was evaluated (FIG. 4E and FIG. 4F). The delivery of proteins such as growth factors has been widely used to enhance wound healing. BSA release from 1% (w/v) alginate was slower in comparison to Rh-B, especially in the initial hours, which could be due to the larger molecular size of the BSA and/or a higher affinity of BSA to the alginate (FIG. 4E).

The compatibility of the platform with another hydrogel was examined through the application of PEGDA as a photocrosslinkable hydrogel. The release of BSA from PEGDA hydrogels was faster than in alginate hydrogels (FIG. 4F), most likely due to the difference between the network electrostatic charge. These findings indicate that the composition of the hydrogel, potentially the network electrostatic charge, as well as the geometrical features of the needles can be optimized to fine-tune controlled drug delivery. However, the fabrication of gels from PEGDA was more challenging due to limited penetration of light into the needles.

Correct dosage is important in drug delivery; an insufficient amount will be ineffective and an excessive dose can cause local and systemic side effects and toxicity. The developed MNA allows different methods for controlling the amount of delivered therapeutic. First, the drug concentration in the hydrogels can be easily adjusted before loading, which determines the total drug mass contained in the MNA. Additionally, the geometry of the array can be customized in terms of needle density, total needle count, and needle size, all of which can be used to control the total volume of hydrogel that will be exposed to the tissue. In this way, the total drug dose can also be controlled. Finally, optimizing a hydrogel delivery profile for a particular drug gives precise control of the drug delivered over time, so the dose is continuously maintained in the effective therapeutic range.

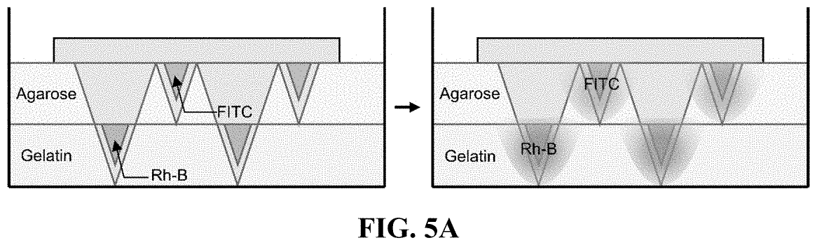

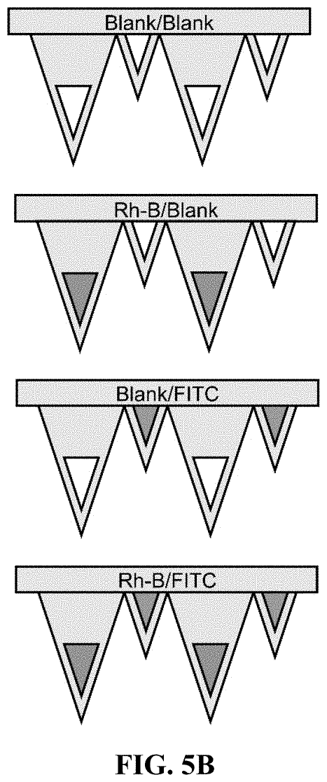

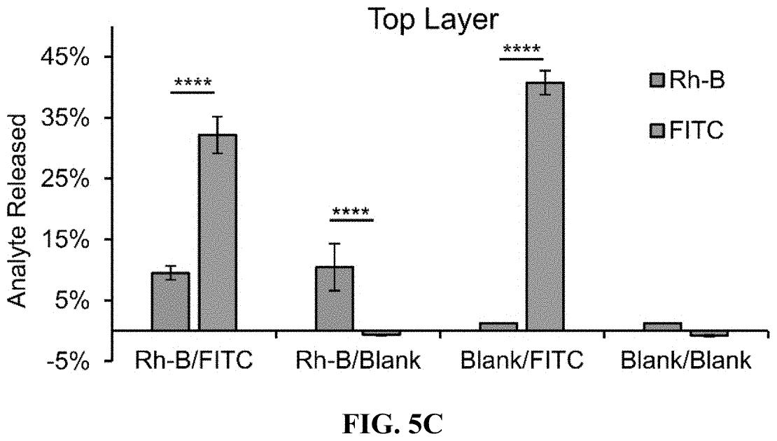

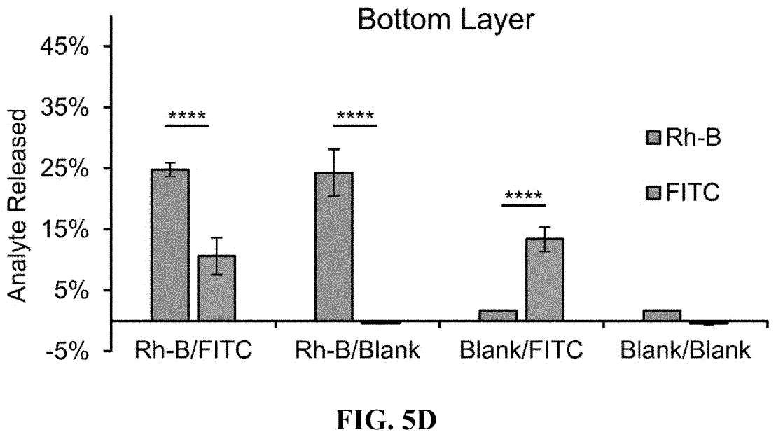

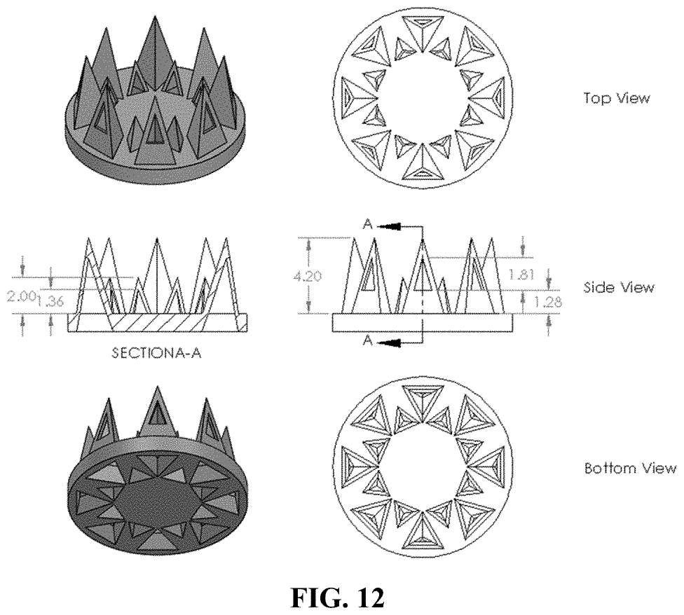

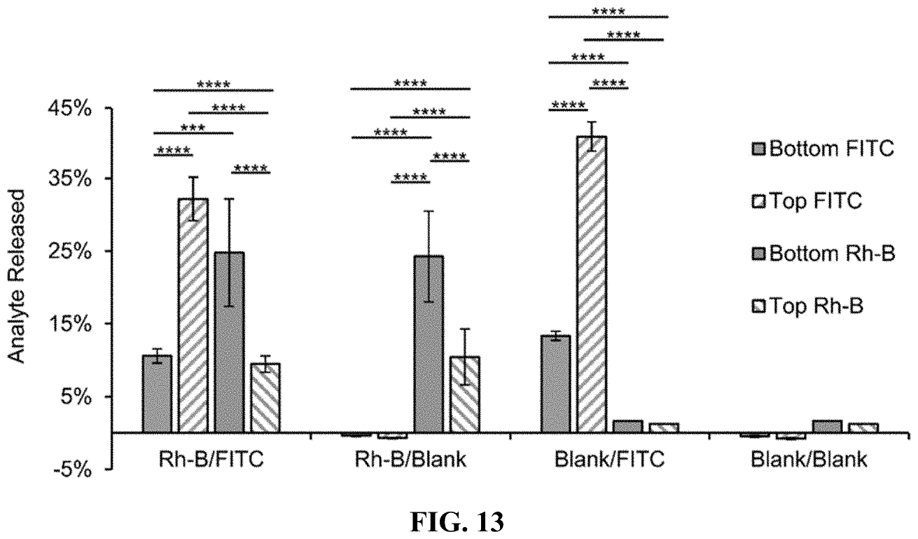

An additional release experiment was conducted to evaluate the performance of the MNA at the dual-delivery of drugs. An alternate design (FIG. 12) employing a wall along the base of the longer needle was utilized to improve drug localization to the desired depth. For this release, alginate mixed with Rh-B was loaded into the longer needles, with BSA-FITC laden alginate in the shorter needles. Rather than releasing into PBS, these arrays were inserted into a two-gel skin model including agarose and gelatin (FIG. 5A). Following the release, the gel layers were separated, melted, and read in a plate reader for their Rh-B and BSA-FITC content. The dual release loading, as well as control conditions, are represented schematically in FIG. 5B. The measured release results are quantified in FIG. 5C, FIG. 5D, and FIG. 13. There was a certain amount of cross-contamination between the layers. Like in real skin, there were no barriers between the layers of gel, allowing eventual diffusion of the released molecules from one layer to another. However, the Rh-B signal is stronger in the gelatin, which made up the bottom layer, showing that the longer needles were able to target drug delivery to the desired position. Correspondingly, there is a greater BSA-FITC signal in the top, agarose layer demonstrating the desired release from the shorter needles. This experiment builds on the delivery distribution results seen in FIG. 1C and FIG. 1D while emphasizing the versatility of 3D printing to customize MNA designs for different applications.

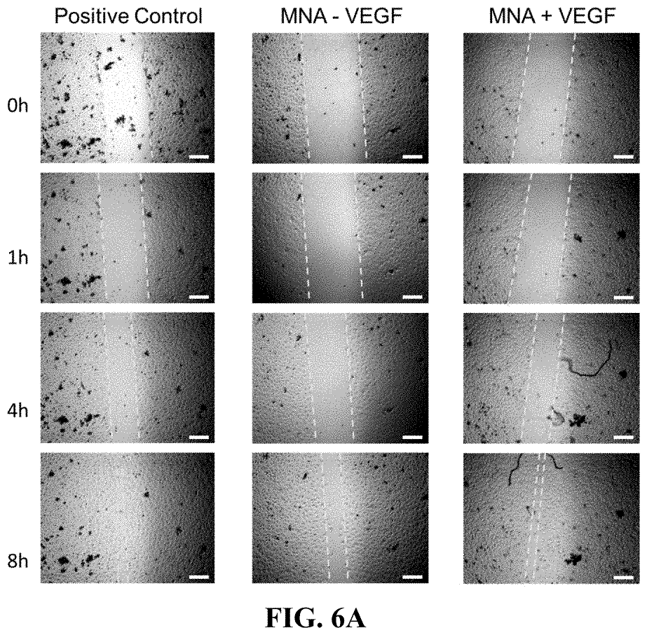

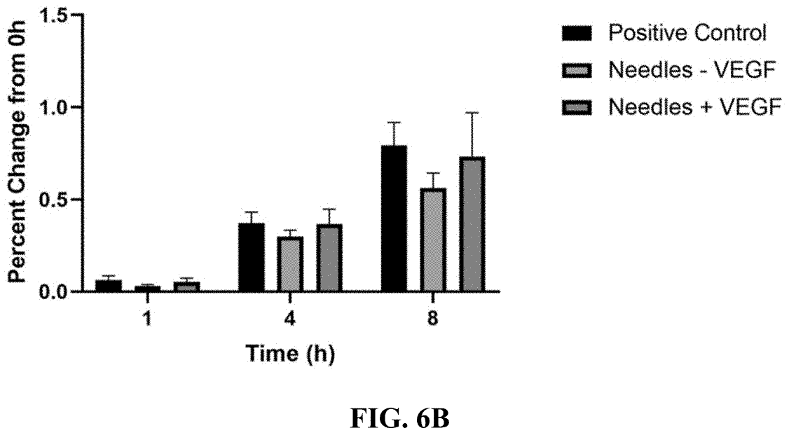

The ability of the platform to preserve the bioactivity of encapsulated therapeutics was assessed by loading vascular endothelial growth factor (VEGF), an important angiogenic factor. VEGF is known to activate the migration of endothelial cells, and its angiogenic activity was investigated by monitoring the rate of endothelial cell migration through a standard scratch assay (FIG. 6A and FIG. 6B). A pipette tip was used to create similarly sized scratches in confluently cultured HUVECs, then the treatments were applied. Three different groups were evaluated: MNA−VEGF, MNA+VEGF, and a positive control. In the positive control group, VEGF was mixed with the culture media to induce HUVEC migration and accelerate gap closure.

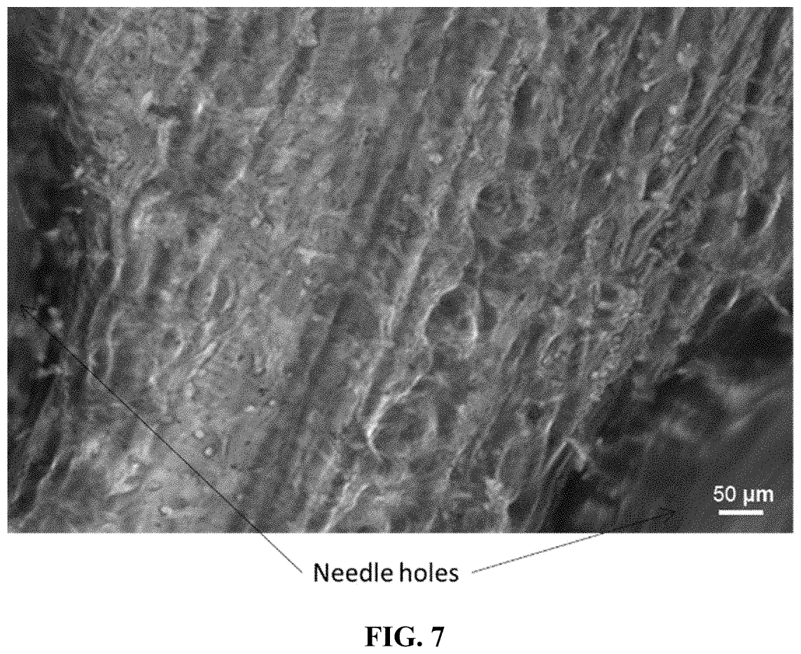

Although the cytocompatibility of the fabricated structure was confirmed using a Live/Dead assay (FIG. 7), the presence of a needle array resting on the cultured cells may affect their growth. Consequently, VEGF-free MNAs were used as a negative control where the MNAs were placed in the culture media without the incorporation of VEGF in the embedded hydrogel. In the MNA+VEGF group, the needles were filled with 1% (w/v) alginate loaded with VEGF and were similarly placed directly in the well, allowing the VEGF to diffuse into the culture media. Representative images of the scratches for each group are shown for each time point (FIG. 6A). As the results show, after 8 h of culture, the MNA+VEGF group exhibited a similar healing potential to the positive control group where cells nearly closed the gap in its entirety. However, the MNA−VEGF group had a slower healing rate and an observable scratch remained at the end of the study.

Although the differences between the groups were not statistically significant, there is a clear trend in the data (FIG. 6B). The healing rate for the MNA+VEGF replicates across all time points was comparable to our positive control, surpassing the MNA−VEGF group. This set of data suggests that VEGF had a positive effect on HUVEC migration. Overall, it can be concluded that the bioactivity of VEGF encapsulated within the gel-filled MNAs was preserved throughout the fabrication and delivery process. Paired with the results seen in FIG. 4A-F and FIG. 5A-D, the platform displays the ability to accurately deliver biologically active therapeutics to targeted levels of tissue.

Disclosed herein is a robust, customizable, and novel transdermal drug delivery platform. The MNA can be 3D-printed in any desired shape or size and filled with different hydrogels that encapsulate relevant bioactive factors. The flexibility of the fabrication method in the formation of multi-length MNAs provides an opportunity to deliver multiple drugs at different target depths. A rapid filling system was developed to improve the production speed of the platform, as this was the rate-limiting step in complete MNA preparation. The needles withstood compression testing past the force required for skin penetration, and past this value they failed by bending rather than breaking, an optimum failure mode for inserted needles. Their ability to penetrate porcine skin and maintain constituent hydrogels intact during insertion and removal was demonstrated. This allows therapeutics to be removed or replaced easily in a non-invasive fashion if conditions change. The capacity of the platform to be filled with different types of hydrogels and deliver various drug models was successfully demonstrated. Finally, the MNAs were found to be effective at containing and releasing bioactive VEGF and promoting gap closure in a HUVEC scratch assay. Collectively, the developed patch is easy-to use and a robust tool for controlling the temporal and spatial distribution of drugs in a passive fashion.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A drug delivery device comprising: a microneedle array comprising a plurality of microneedles on a surface of a substrate, each microneedle comprising a core comprising a hydrogel; and a layer on a surface of the core, the layer comprising a material having a compressive modulus of at least 1 MPa.

Clause 2. The drug delivery device of clause 1, wherein the hydrogel comprises poly(ethylene glycol diacrylate), poly (l-lactic acid) (PLLA), poly(d,l-lactide-co-glycolide) (PLGA), polycaprolactone (PCL), polyglycolic acid (PGA), polyvinyl alcohol, polyacrylic acid, poly(N-isopropylacrylamide), gelatin methacryloyl, alginate, collagen, gelatin, fibrin, hyaluronic acid, silk, blood derived materials, poly(N-isopropylacrylamide) (PNIPAm), or a combination thereof.

Clause 3. The drug delivery device of clause 1 or 2, wherein the material comprises a polymer or a metal.

Clause 4. The drug delivery device of any one of clauses 1-3, wherein the polymer is a printable polymer for stereolithography or digital light processing 3D printing.

Clause 5. The drug delivery device of any one of clauses 1-4, wherein the hydrogel comprises a biologically active agent.

Clause 6. The drug delivery device of any one of clauses 1-5, wherein the hydrogel comprises a plurality of nanoparticles.

Clause 7. The drug delivery device of any one of clauses 1-6, wherein the biologically active agent comprises a nucleotide, a polynucleotide, a protein, a peptide, a polypeptide, a carbohydrate, a lipid, a small molecule drug, a cell, or a combination thereof.

Clause 8. The drug delivery device of any one of clauses 1-7, wherein the hydrogel has a plurality of pores, each pore having a diameter of about 10 nm to about 250 μm.

Clause 9. The drug delivery device of any one of clauses 1-8, wherein each microneedle has a length of about 100 μm to about 5,000 μm.

Clause 10. The drug delivery device of any one of clauses 1-9, wherein each microneedle comprises a first end that is coupled to the substrate, a second end opposite the first end, and a channel extending therethrough from the first end to the second end, and wherein the hydrogel is positioned within the channel.

Clause 11. The drug delivery device of any one of clauses 1-10, wherein each microneedle has a failure point at about 12% strain to about 20% strain as measured by jaw displacement.

Clause 12. The drug delivery device of any one of clauses 1-11, wherein each microneedle does not break at its failure point.

Clause 13. The drug delivery device of any one of clauses 1-12, wherein the substrate comprises at least two different materials, and wherein one of the materials is the same composition as the layer.

Clause 14. The drug delivery device of any one of clauses 1-13, wherein the substrate is configured for the plurality of microneedles to penetrate and conform to a biological topography.

Clause 15. The drug delivery device of any one of clauses 1-14, wherein the array comprises a first plurality of microneedles, wherein each microneedle of the first plurality has a first length; and a second plurality of microneedles, wherein each microneedle of the second plurality has a second length, the second length being different than the first length.

Clause 16. The drug delivery device of clause 15, wherein the first plurality of microneedles comprises a first biologically active agent and the second plurality of microneedles comprises a second biologically active agent.

Clause 17. A method of making a microneedle array, the method comprising: printing a microneedle array, the microneedle array comprising a plurality of microneedles on a surface of a substrate, each microneedle comprising a first end that is coupled to the substrate, a second end opposite the first end, and a channel extending therethrough from the first end to the second end; adding a prepolymer to the channel of each microneedle; and crosslinking the prepolymer to provide a hydrogel in the channel of each microneedle.

Clause 18. The method of clause 17, wherein the microneedle array is washed with a base solution prior to addition of the prepolymer.

Clause 19. The method of clause 17 or 18, wherein the prepolymer is added to each microneedle via a loading device, the loading device comprising a base having a first end and a second end, a plurality of outlets coupled to the first end of the base that align in number and arrangement with the plurality of microneedles, and an outlet coupled to the second end of the base in fluid communication with the plurality of outlets.

Clause 20. A method of treating a disorder in a subject in need thereof, the method comprising contacting an area of the subject's skin with the drug delivery device of any one of clauses 5-16, wherein the biologically active agent is transdermally delivered to the subject.

Clause 21. The method of clause 20, wherein the drug delivery device delivers at least two different biologically active agents to the subject.

Clause 22. The method of clause 20 or 21, wherein the disorder comprises burns, diabetic ulcers, chronic wounds, muscle abnormalities, melanoma, cancer immunotherapy, or keloids.

What is claimed is:

1. A drug delivery device comprising:
a microneedle array comprising a plurality of microneedles on a surface of a substrate, each microneedle comprising
a core comprising a hydrogel; and
a layer on a surface of the core, the layer comprising a material having a compressive modulus of at least 1 MPa.

2. The drug delivery device of claim 1, wherein the hydrogel comprises poly(ethylene glycol diacrylate), poly (l-lactic acid) (PLLA), poly(d,l-lactide-co-glycolide) (PLGA), polycaprolactone (PCL), polyglycolic acid (PGA), polyvinyl alcohol, polyacrylic acid, poly(N-isopropylacrylamide), gelatin methacryloyl, alginate, collagen, gelatin, fibrin, hyaluronic acid, silk, blood derived materials, poly(N-isopropylacrylamide) (PNIPAm), or a combination thereof.

3. The drug delivery device of claim 1, wherein the material comprises a polymer or a metal.

4. The drug delivery device of claim 1, wherein the polymer is a printable polymer for stereolithography or digital light processing 3D printing.

5. The drug delivery device of claim 1, wherein the hydrogel comprises a biologically active agent.

6. The drug delivery device of claim 1, wherein the hydrogel comprises a plurality of nanoparticles.

7. The drug delivery device of claim 5, wherein the biologically active agent comprises a nucleotide, a polynucleotide, a protein, a peptide, a polypeptide, a carbohydrate, a lipid, a small molecule drug, a cell, or a combination thereof.

8. The drug delivery device of claim 1, wherein the hydrogel has a plurality of pores, each pore having a diameter of about 10 nm to about 250 μm.

9. The drug delivery device of claim 1, wherein each microneedle has a length of about 100 μm to about 5,000 μm.

10. The drug delivery device of claim 1, wherein each microneedle comprises a first end that is coupled to the substrate, a second end opposite the first end, and a channel extending therethrough from the first end to the second end, and wherein the hydrogel is positioned within the channel.

11. The drug delivery device of claim 1, wherein each microneedle has a failure point at about 12% strain to about 20% strain as measured by jaw displacement.

12. The drug delivery device of claim 1, wherein each microneedle does not break at its failure point.

13. The drug delivery device of claim 1, wherein the substrate comprises at least two different materials, and wherein one of the materials is the same composition as the layer.

14. The drug delivery device of claim 1, wherein the substrate is configured for the plurality of microneedles to penetrate and conform to a biological topography.

15. The drug delivery device of claim 1, wherein the array comprises
a first plurality of microneedles, wherein each microneedle of the first plurality has a first length; and
a second plurality of microneedles, wherein each microneedle of the second plurality has a second length, the second length being different than the first length.

16. The drug delivery device of claim 15, wherein the first plurality of microneedles comprises a first biologically active agent and the second plurality of microneedles comprises a second biologically active agent.

17. A method of making a microneedle array, the method comprising:
printing a microneedle array, the microneedle array comprising a plurality of microneedles on a surface of a substrate, each microneedle comprising a first end that is coupled to the substrate, a second end opposite the first end, and a channel extending therethrough from the first end to the second end;
adding a prepolymer to the channel of each microneedle; and
crosslinking the prepolymer to provide a hydrogel in the channel of each microneedle.

18. The method of claim 17, wherein the microneedle array is washed with a base solution prior to addition of the prepolymer.

19. The method of claim 17, wherein the prepolymer is added to each microneedle via a loading device, the loading device comprising
a base having a first end and a second end,
a plurality of outlets coupled to the first end of the base that align in number and arrangement with the plurality of microneedles, and
an outlet coupled to the second end of the base in fluid communication with the plurality of outlets.

20. A method of treating a disorder in a subject in need thereof, the method comprising contacting an area of the subject's skin with the drug delivery device of claim 5, wherein the biologically active agent is transdermally delivered to the subject.

21. The method of claim 20, wherein the drug delivery device delivers at least two different biologically active agents to the subject.

22. The method of claim 20, wherein the disorder comprises burns, diabetic ulcers, chronic wounds, muscle abnormalities, melanoma, cancer immunotherapy, or keloids.
